(19) United States
(12) Patent Application Publication
(10) Pub. No.: US 2021/0241908 A1
(43) Pub. Date: Aug. 5, 2021

(54) MULTI-SENSOR BASED HMI/AI-BASED SYSTEM FOR DIAGNOSIS AND THERAPEUTIC TREATMENT OF PATIENTS WITH NEUROLOGICAL DISEASE

(71) Applicant: MINDMAZE HOLDING SA, Lausanne (CH)

(72) Inventors: Martin CIUPA, Lausanne (CH); Tej TADI, Lausanne (CH)

(21) Appl. No.: 17/050,702

(22) PCT Filed: Apr. 24, 2019
(86) PCT No.: PCT/IB2019/053391

§ 371 Date: Oct. 26, 2020

Related U.S. Application Data
(60) Provisional application No. 62/663,173, filed on Apr. 26, 2018.
(60) Provisional application No. 62/824,953, filed on Mar. 27, 2019.

(51) Int. Cl. G16H 50/20; G06N 20/00; G06F 17/14; G16H 50/70; G16H 10/60; G16H 20/00; A61B 5/00; A61B 5/1171; A61B 5/369; A61B 5/11; A61B 5/389; A61B 5/318; A61B 6/03; A61B 5/055

(52) U.S. Cl. CPC G16H 50/20; A61B 5/7275; G06F 17/142; G06F 17/147; G16H 50/70; G16H 10/60; G16H 20/00; A61B 5/7257; A61B 5/726; A61B 5/7264; A61B 5/1176; A61B 5/369; A61B 5/4803; A61B 5/1116; A61B 5/389; A61B 5/318; A61B 6/037; A61B 5/055; A61B 6/032; A61B 5/7203; A61B 5/4094; A61B 5/4088; A61B 5/4082; G06N 20/00

(57) ABSTRACT

A system and method for diagnosis and therapeutic treatment of patients with neurological disease from a plurality of sensors, and in particular, to such a system, method and apparatus for analyzing data from a plurality of sensors with an AI (artificial intelligence) algorithm.

### FIELD OF THE DISCLOSURE

The present invention is of a system, method, and apparatus for diagnosis and therapeutic treatment of patients with neurological disease from a plurality of sensors, and in particular, to such a system, method and apparatus for analyzing data from a plurality of sensors with an AI (artificial intelligence) algorithm.

### BACKGROUND

There are multiple considerations for diagnosis and treatment of neurological diseases. For example, different diseases may have widely different prognoses, depending upon the initial condition of the subject suffering from the disease. Other such diseases may have a constellation of causes which can also lead to highly variable outcomes.

It would be highly desirable to be able to connect the results of diagnostic tests more directly to the best treatment regimen for the patient, for more personalized medicine. It would also be highly desirable to be able to monitor the patient as they receive treatment, to make any necessary adjustments to obtain the best outcomes.

### SUMMARY OF AT LEAST SOME EMBODIMENTS

According to at least some embodiments, there is provided a system and method for diagnosis and therapeutic treatment of patients with neurological disease from a plurality of sensors, and in particular, to such a system, method and apparatus for analyzing data from a plurality of sensors with an AI (artificial intelligence) algorithm.

Preferably, multiple sensor signals are combined to determine the prognosis for the patient through an AI engine or system. Also preferably, such signals are measured a plurality of times during treatment, to determine whether the treatment is beneficial for the patient.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the apparatuses, devices, methods, and systems of the present disclosure involve performing or completing specific selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be performed as a number of software instructions being executed by a computer (e.g., a processor of the computer) using an operating system. In any case, selected steps of methods of at least some embodiments of the disclosure can be described as being performed by a processor, such as a computing platform for executing a plurality of instructions.

Software (e.g., an application, computer instructions) which is configured to perform (or cause to be performed) specific functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality. Furthermore, the phrase “abstraction layer” or “abstraction interface,” as used with some embodiments, can refer to computer instructions (which can be a set of instructions, an application, software) which are operable on a computational device (as noted, e.g., a processor) to cause the computational device to conduct and/or achieve one or more specific functionality. The abstraction layer may also be a circuit (e.g., an ASIC) to conduct and/or achieve one or more specific functionality. Thus, for some embodiments, and claims which correspond to such embodiments, the noted feature/functionality can be described/claimed in a number of ways (e.g., abstraction layer, computational device, processor, module, software, application, computer instructions, and the like).

Some embodiments are described concerning a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

Embodiments of the present disclosure herein described are by way of example only, with reference to the accompanying drawings. With specific reference now to the drawings in detail, it is stressed that the particulars shown are by way of example and for purposes of illustrative discussion of some embodiments of the present invention only, and are presented in order to provide what is believed to be the most useful and readily understood description of the principles and conceptual aspects of some of the embodiments. In this regard, no attempt is made to show details of some embodiments in more detail than is necessary for a fundamental understanding thereof.

FIG. 1A shows a non-limiting example of a system according to at least some embodiments of the present disclosure;

FIG. 1B shows an exemplary non-limiting method for diagnosis and treatment using the system as described herein according to at least some embodiments;

FIG. 2 shows a non-limiting exemplary diagnostic flow for implementation with the system and apparatuses as described herein;

FIG. 3A shows an exemplary, illustrative non-limiting tracking engine, optionally for use with the system of FIG. 1 or the method of FIG. 2, according to at least some embodiments of the present invention;

FIG. 3B shows an exemplary, illustrative non-limiting method for tracking the user, optionally performed with the system of FIG. 1 or 3A, according to at least some embodiments of the present disclosure;

FIG. 4A illustrates an example system for acquiring and analyzing EMG signals, according to at least some embodiments;

FIG. 4B shows an exemplary, non-limiting, illustrative method for facial expression classification according to at least some embodiments;

FIG. 5 shows a non-limiting exemplary EEG flow;

FIG. 6 describes a non-limiting exemplary optical micro expression flow;

FIG. 7 describes an exemplary non-limiting flow for analyzing the voice of the user;

FIG. 8 relates to a non-limiting exemplary convolutional and pooling neural network implemented as a CNN (convolutional neural network) with combined connected layers as a non-limiting example of a neural network that may be used with the present invention;

FIG. 9A relates to a non-limiting exemplary flow for analysis and treatment;

FIG. 9B relates to a non-limiting expanded feedback loop for diagnosis and optional therapeutic intervention;

FIG. 10 relates to a non-limiting exemplary neural disease diagnostic classification method;

FIG. 11 relates to a non-limiting exemplary analysis flow for treatment;

FIG. 12 relates to a non-limiting exemplary method for converting image data to a Hilbert space;

FIG. 13 relates to a non-limiting exemplary method for transforming EEG data into the Hilbert space, by using the Hilbert transform;

FIGS. 14A and 14B relate to non-limiting exemplary flows for applying the Hilbert space or the Hilbert transform to ECG data;

FIG. 15 relates to an exemplary, non-limiting method for the treatment of a subject suffering from Parkinson's disease;

FIGS. 16A and 16B relate to exemplary processes for the treatment of a subject suffering from a stroke; and

FIG. 17 relates to an exemplary process for the treatment of a subject suffering from multiple sclerosis (MS).

### DESCRIPTION OF AT LEAST SOME EMBODIMENTS

Various neurological indications may be analyzed to determine a prognosis and also the effect of therapy on the indication. Non-limiting examples of such additional neurological indications include dementia, epilepsy, headache disorders, multiple sclerosis, neuroinfection, neurological disorders associated with malnutrition, pain associated with neurological disorders, Parkinson's disease, stroke, amyotrophic lateral sclerosis (ALS), post-traumatic stress disorder (PTSD) and traumatic brain injuries.

Data is preferably obtained from a plurality of different sensor types as described herein. Preferably also data from imagining modalities is obtained, along with data from medical records and other unstructured information. Other non-limiting examples of data include facial expression data, voice data, camera data and motion data. Motion data may be obtained for example by tracking one or more motions of a user, for example through analysis of optical data.

The data is then preferably then analyzed through being transformed to a single data space. As a non-limiting example, the data may be vectorized to a common data space, such as Hilbert space for example. After being transformed to a single data space, the data may be analyzed through a neural net or classical machine learning algorithm for example.

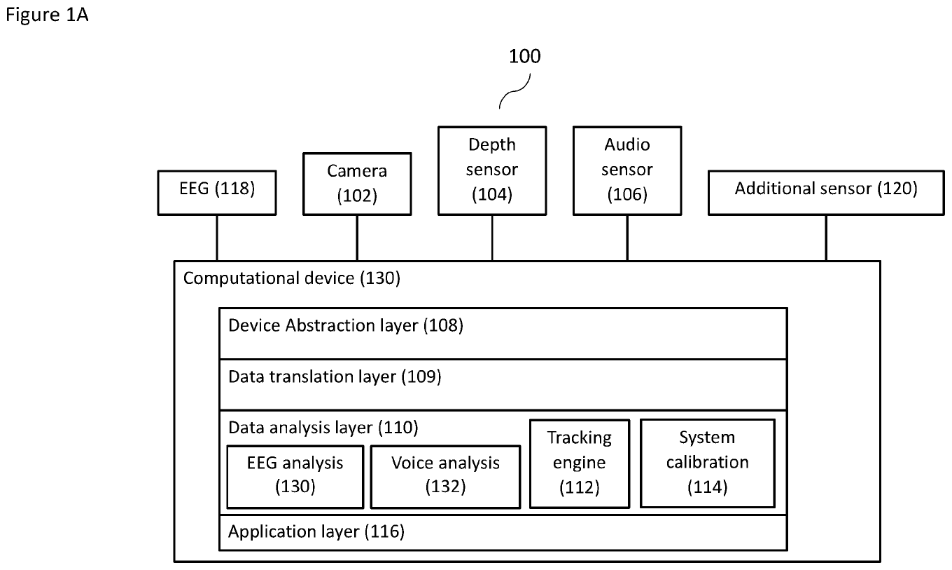

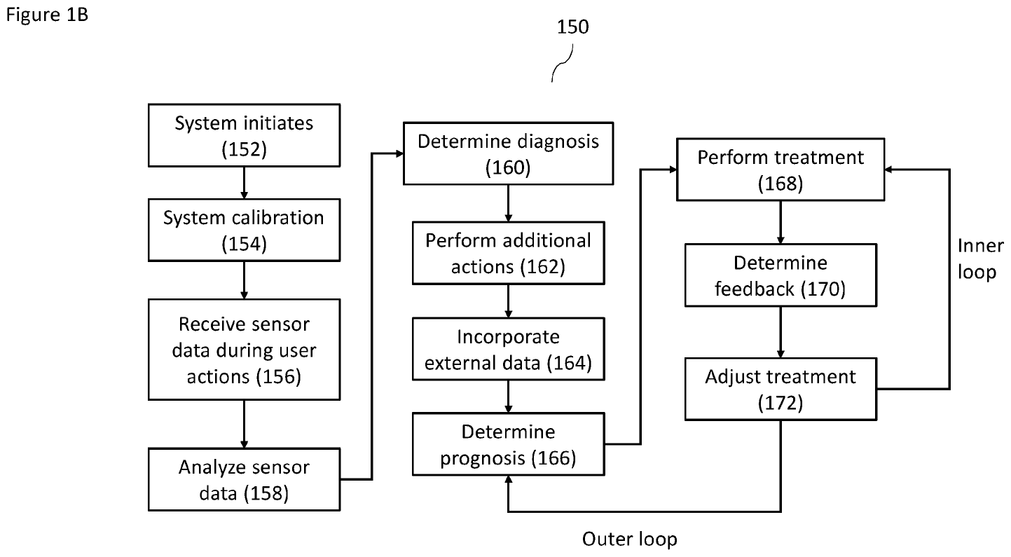

FIG. 1A shows a non-limiting example of a system according to at least some embodiments of the present disclosure. As shown, a system 100 features a camera 102, a depth sensor 104 and optionally an audio sensor 106. Optionally an additional sensor 120 is also included. Optionally camera 102 and depth sensor 104 are combined in a single product (e.g., Kinect® product of Microsoft®, and/or as described in U.S. Pat. No. 8,379,101). FIG. 1B shows an exemplary implementation for camera 102 and depth sensor 104. Optionally, camera 102 and depth sensor 104 can be implemented with the LYRA camera of Mindmaze SA. The integrated product (i.e., camera 102 and depth sensor 104) enables, according to some embodiments, the orientation of camera 102 to be determined with respect to a canonical reference frame. Optionally, three or all four sensors (e.g., a plurality of sensors) are combined in a single product.

The sensor data, in some embodiments, relates to physical actions of a user (not shown), which are accessible to the sensors. For example, camera 102 can collect video data of one or more movements of the user. Camera 102 may also be used to detect pulse rate through changes in skin color. Depth sensor 104 may provide data to determine the three-dimensional location of the user in space according to the distance of the user from depth sensor 104 (or more specifically, the plurality of distances that represent the three-dimensional volume of the user in space). Depth sensor 104 can provide TOF (time of flight) data regarding the position of the user, which, when combined with video data from camera 102, allows a three-dimensional map of the user in the environment to be determined. As described in greater detail below, such a map enables the physical actions of the user to be accurately determined, for example, with regard to gestures made by the user. Audio sensor 106 preferably collects audio data regarding any sounds made by the user, optionally including, but not limited to, speech. In some embodiments, this audio sensor can be used for vocal interaction with speech synthesis, for example, providing audio instructions to collect particular vocal interactions (e.g., particular types of answers, words, and the like) or evoking particular types of responses, including with regard to audio commands to system 100. An EEG (electroencephalography) sensor 118, typically implemented as a plurality of such sensors, preferably collects EEG signals.

Additional sensor 120 can collect biological signals about the user and/or may collect additional information to assist the depth sensor 104. Non-limiting examples of biological signals include a heartrate sensor, an oxygen saturation sensor, infrared pulse sensor, optical pulse sensor, an EKG or EMG sensor, or a combination thereof.

Sensor signals are collected by a device abstraction layer 108, which preferably converts the sensor signals into data which is sensor-agnostic. Device abstraction layer 108 preferably handles the necessary preprocessing such that, if different sensors are substituted, only changes to device abstraction layer 108 would be required; the remainder of system 100 can continue functioning without changes (or, in some embodiments, at least without substantive changes). Device abstraction layer 108 preferably also cleans signals, for example, to remove or at least reduce noise as necessary, and can also be used to normalize the signals. Device abstraction layer 108 may be operated by a computational device (not shown), and any method steps may be performed by a computational device (note—modules and interfaces disclosed herein are assumed to incorporate, or to be operated by, a computational device, even if not shown).

The preprocessed signal data from the sensors are preferably then be passed to a data translation layer 109, which transforms the data into an appropriate structure and/or data space, for example and without limitation by applying one or more transforms. Non-limiting examples of such transforms include FFT, DWT (discrete wavelet transform) and Hilbert space transforms. Preferably the translated data is suitable for input to a machine learning algorithm as described in greater detail below. Optionally data translation layer 109 is implemented as a plurality of split translation layers, for greater benefits of security, for example by restricting access to different aspects of the data through separate encryption of the split translation layers.

Next the translated data is preferably fed to a data analysis layer 110, which preferably performs data analysis on the sensor data for consumption by an application layer 116 (according to some embodiments, “application,” means any type of interaction with a user). Preferably, such analysis includes tracking analysis, performed by a tracking engine 112, which can track the position of the user's body and can also track the position of one or more body parts of the user, including but not limited, to one or more of arms, legs, hands, feet, head and so forth. Tracking engine 112 can analyze the preprocessed signal data to decompose physical actions made by the user into a series of gestures. A “gesture” in this case may include an action taken by a plurality of body parts of the user, such as taking a step while swinging an arm, lifting an arm while bending forward, moving both arms, and so forth. Such decomposition and gesture recognition can also be done separately, for example, by a classifier trained on information provided by tracking engine 112 with regard to tracking the various body parts.

It is noted that while the term “classifier” is used throughout, this term is also intended to encompass “regressor”. For machine learning, the difference between the two terms is that for classifiers, the output or target variable takes class labels (that is, is categorical). For regressors, the output variable assumes continuous variables (see for example http://scottge.net/2015/06/12/ml101-regressoin-vs-classification-vs-clustering-problems/).

The tracking of the user's body and/or body parts, optionally decomposed to a series of gestures, can then be provided to application layer 116, which translates the actions of the user into a type of reaction and/or analyzes these actions to determine one or more action parameters. For example, and without limitation, a physical action taken by the user to lift an arm is a gesture which could translate to application layer 116 as lifting a virtual object. Alternatively, or additionally, such a physical action could be analyzed by application layer 116 to determine the user's range of motion or ability to perform the action. In situations where data sets are limited, classical machine learning techniques such as Naïve Bayesian algorithms may be more efficient (in terms of requiring less datasets for training) or useful. However, in a more data rich environment, advanced neural net technology, with supervised or unsupervised architectures, may be more useful.

Application layer 116 also preferably performs one or more diagnostic functions as described in greater detail below, for example to diagnose a brain injury or disease.

Data analysis layer 110, in some embodiments, includes a system calibration module 114. As described in greater detail below, system calibration module 114 is configured to calibrate the system with respect to the position of the user, in order for the system to track the user effectively. System calibration module 114 can perform calibration of the sensors with respect to the requirements of the operation of application layer 116 (although, in some embodiments—which can include this embodiment—device abstraction layer 108 is configured to perform sensor specific calibration). Optionally, the sensors may be packaged in a device (e.g., Microsoft® Kinect), which performs its own sensor specific calibration.

Data analysis layer 110, in some embodiments, includes an EEG analysis module 130 and a voice analysis module 132. EEG analysis module 130 preferably analyzes EEG signals while voice analysis module 132 analyzes voice signals. Such signal analysis is also optionally combined as described in greater detail below.

FIG. 1B shows an exemplary non-limiting method for diagnosis and treatment using the system as described herein according to at least some embodiments. As shown in a flow 150, the process begins when the system initiates at 152, and system calibration is then performed at 154. After calibration, user data is preferably collected by receiving sensor data during actions performed by the user in 156. The sensor data is analyzed in 158, and the diagnosis is determined in 160. Preferably one or more additional actions are performed in 162 to be able to obtain additional data. Additionally, or alternatively, additional sensor data may be obtained, for example from biosignals.

Next, external data is incorporated in 164 such as various types of data, including but not limited to other biosignal data, fMRI data, other types of image data, and unstructured data such as doctor's notes and the like. Next, a prognosis is determined in 166. A treatment is performed in 168 and then feedback from the treatment is determined in 170, for example by performing another diagnostic test or tests, obtaining additional user data and so forth. Optionally, the feedback is cybernetic feedback.

The treatment is then adjusted in 172. Preferably, steps 168 to 172 form an inner loop which may be performed repeatedly. Optionally, then an outer loop would include performing treatment in 168, determining feedback and 170, adjusting treatment in 172 and then reconsidering the prognosis and 166. Optionally, other steps that were previously performed such as incorporating external data may also be performed begin as part of the outer loop.

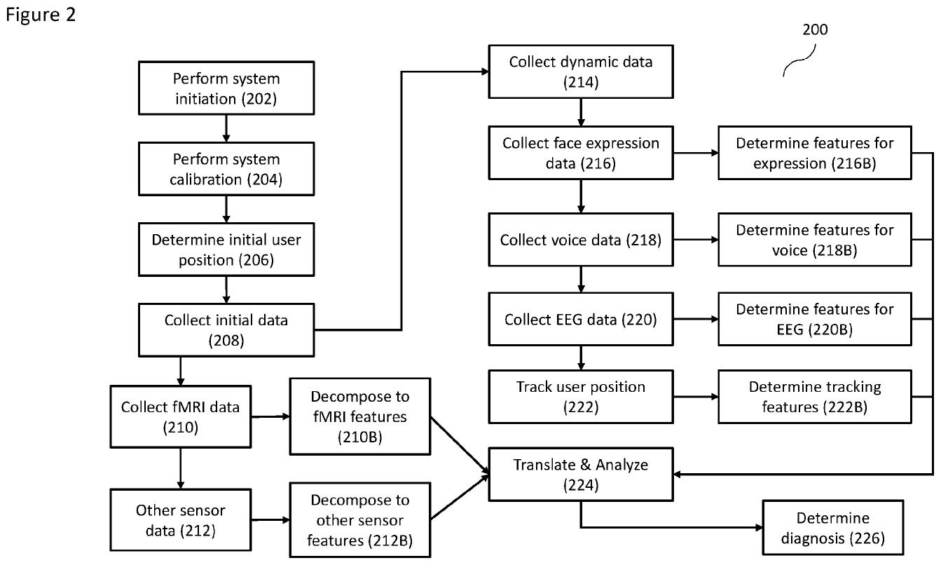

FIG. 2 shows a non-limiting exemplary diagnostic flow for implementation with the system and apparatuses as described herein. In the flow 200, the system initiates at 202, and system calibration is performed at 204 as previously described. Next, the initial user position is determined at 206 if tracking of a user action is to be performed and the initial data is collected at 208. Preferably then the method branches. In one branch, fMRI data is collected in 210 and optionally, other sensor data is collected in 212. The fMRI data at 210 is preferably decomposed to one or more fMRI features in 210B. For example and without limitation fMRI data may be decomposed through image analysis, for example to detect one or more lesions. If one or more lesions are detected, the lesion volume and location may be determined. Both lesion volume and location may have an effect on the expected prognosis. A non-limiting example of a method for doing so is described in U.S. Pat. No. 9,265,441, assigned to Siemens Healthcare GmbH, published on Feb. 23 2016. Another non-limiting example is given in U.S. Pat. No. 9,208,557, assigned to Olea Medical SA, published on Dec. 8 2015. This patent describes a method for analyzing an artery to determine whether a blockage is present and the effect on arterial output. The method may be used for estimating hemodynamic parameters by applying soft probabilistic methods to perfusion imaging. The other sensor data collected at 212 is preferably decomposed into other sensor features at 212b.

Optionally the fMRI data and the sensor data are all collected simultaneously but may be collected sequentially. The fMRI features and other sensor features are preferably fed into step 224 for translation analysis. Turning back to the other branch, from initial data collection 208, preferably dynamic data is collected in 214. Dynamic data 214 is preferably collected during one or more user movements or during one or more actions performed by the user.

Next phase, expression data is collected in 216, voice data is collected at 218, and the EEG data is collected at 220. Optionally steps 214 to 220 are performed simultaneously but may also be performed sequentially. Face expression data 216 is then preferably used to determine features for expression such as classification as previously described in 216B. Voice data is then collected in 218 to determine voice features in 218B. EEG data in 220 is then used to determine features for the EEG 220B as described in greater detail below. Simultaneously or sequentially, while the data is being collected or after, the user position is tracked in 222 and then tracking features are determined in 222B. All of this information, these different types of features are preferably fed to translate and analyze step 224, which is then used to determine the diagnosis in 226.

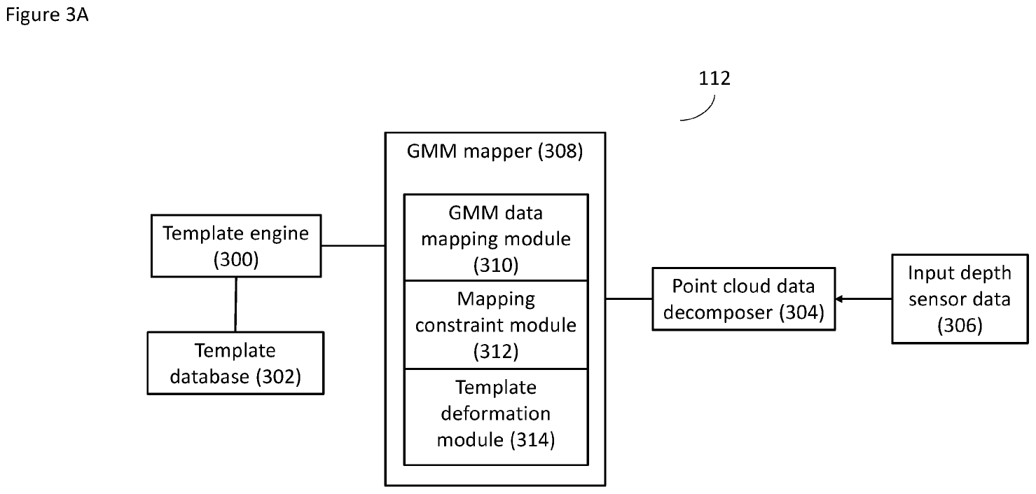

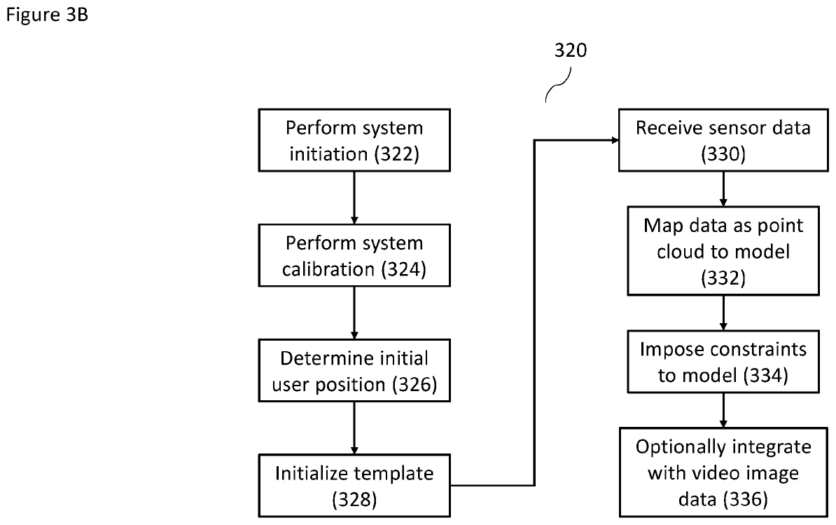

FIG. 3A shows an exemplary, illustrative non-limiting tracking engine, optionally for use with the system of FIG. 1 or the method of FIG. 2, according to at least some embodiments of the present invention. For this embodiment of the tracking engine, the data is assumed to be mapped to a GMM, but as described herein, optionally a classifier is used instead. As shown, the tracking engine features a template engine 300, which reads a template from a template database 302, and then feeds the template to a GMM mapper 308. GMM mapper 308 also receives point cloud information from a point cloud decomposer 304, which receives the depth sensor data as an input in 306. Optionally color camera data could also be provided to point cloud decomposer 304. For example, stereo RGB could be used to assist with the assignment of points to body parts and/or to improve the depth sensor data. Solutions to the problem of configuring depth sensor data to a point cloud is well known in the art and could optionally be performed according to any suitable method. One non-limiting example of a suitable method is provided in “Alignment of Continuous Video onto 3D Point Clouds” by Zhao et al., available at https://pdfs.semanticscholar.org/124c.0ee6a3730a9266dae59d94a90124760fla5c.pdf.

To increase the speed of processing, the depth sensor data may be configured as follows. To do so a KD-tree of the scene each frame is built, so that when computing correspondences from vertices to cloud one only uses the K nearest neighbors and assume a zero-posterior for the rest. As a consequence, the algorithm runs several orders of magnitude faster. The gating of correspondences allows sparsification of both the distance and the posterior matrix with huge gains on computation speed.

As compared to “Real-time Simultaneous Pose and Shape Estimation for Articulated Objects Using a Single Depth Camera” by Mao Ye and Ruigang Yang, IEEE Transactions on Pattern Analysis & Machine Intelligence, 2016, vol. 38, Issue No. 08., which reached real time performance only with a GPU (graphics processing unit), the presently described algorithm, according to some embodiments, can reach real-time performance (100+ fps in a i7 processor) with CPU (central processing unit) only, which is a significant computational advantage.

GMM mapper 308 features a GMM data mapping module 310, a mapping constraint module 312 and a template deformation module 314. GMM data mapping module 310 receives the point cloud data from point cloud decomposer 304 and maps this data onto the GMM, as adjusted by the input template from template engine 300. Next one or more constraints from mapping constraint module 312, for example regarding the angle range that body parts of the user can assume, are applied to the mapped data on the GMM by mapping constraint module 312. Optionally, such information is augmented by deforming the template according to information from template deformation module 314; alternatively, such deformations are applied on the fly by GMM data mapping module 310 and mapping constraint module 312. In this case, template deformation module 314 is either absent or alternatively may be used to apply one or more heuristics, for example according to pose recovery as described in greater detail below.

FIG. 3B shows an exemplary, illustrative non-limiting method for tracking the user, optionally performed with the system of FIG. 1 or 3A, according to at least some embodiments of the present disclosure. As shown in a method 320, at 322, the system initiates activity, for example, by being powered up (i.e., turned on). The system can be implemented as described in FIG. 1 but may also optionally be implemented in other ways. At 324, the system performs system calibration, which can include determining license and/or privacy features. System calibration may also optionally include calibration of one or more functions of a sensor, for example, as described in reference to FIG. 1A.

At 326, an initial user position is determined, which (in some embodiments), is the location and orientation of the user relative to the sensors (optionally at least with respect to the camera and depth sensors). For example, the user may be asked to or be placed such that the user is in front of the camera and depth sensors. Optionally, the user may be asked to perform a specific pose, such as the “T” pose for example, in which the user stands straight with arms outstretched, facing the camera. The term “pose” relates to position and orientation of the body of the user.

At 328 the template is initialized. As described in greater detail below, the template features a model of a human body, configured as only a plurality of parameters and features, such as a skeleton, joints and so forth, which are used to assist in tracking of the user's movements. At 330, sensor data is received, such as for example, one or more of depth sensor data and/or camera data. At 332 and 334, the sensor data is analyzed to track the user, for example, regarding the user's movements. Optionally, the sensor data can be mapped onto a body model, e.g., the body model features an articulated structure of joints and a skin defined by a mesh of vertices that are soft-assigned to the joints of the model with blending weights. In this way, the skin can deform accordingly with the body pose to simulate a realistic human shape.

Optionally, the sensor data is analyzed by mapping onto a GMM (Gaussian mixture model) as described herein. As described in greater detail below, optionally, a classifier can be used. Because the user's pose is not likely to change significantly between frames, optionally, the process at 332 (mapping to a point cloud) and 334 (applying constraints to the model), while performed iteratively, can only performed with regard to a limited number of iterations. For example, the present inventors have found that, surprisingly, as few as 3-10 iterations may be used to map the data. If a GMM is used, each vertex of the skin defines an isotropic gaussian, whose mean location in the 3D space is a function of the rotation parameters of the joints to which the vertex is attached (rotating the left wrist won't affect the position of the vertices on the right-hand skin).

The body model preferably features a sparse-skin representation. Having a sparse-skin representation is convenient to handle occlusions. Both self-occlusions or occlusions of body parts due to clutter or because the user exits the camera frame. One dynamically enables or disables the gaussians that are occluded at a given frame, so that those disabled won't influence the optimization.

In a different direction, it is also straightforward to model amputee users by suppressing the corresponding gaussians. This can be done online during a calibration process or having a therapist manually configuring the body model. At 332, if a GMM is used, the sensor data is mapped as a point cloud to the GMM. The GMM and mapping are optionally implemented as described with regard to “Real-time Simultaneous Pose and Shape Estimation for Articulated Objects Using a Single Depth Camera” by Mao Ye and Ruigang Yang, IEEE Transactions on Pattern Analysis & Machine Intelligence, 2016, vol. 38, Issue No. 08. In this paper, an energy function is described, which is minimized according to the mapping process.

Optionally, only the depth sensor data is used, but alternatively, both the depth sensor and the camera data are used. For example, the calculations may be performed as follows. Given a set of N points x∈X it is desired to fit a GMM with M components (vm).

$$p ( {x}_{n} ) = ( 1 - u ) {\sum }_{m = 1}^{M} p ( {v}_{m} ) p ( {x}_{n} \vert {v}_{m} ) + u \frac{1}{N} \qquad ( 1 )$$

At 334, one or more constraints are imposed on the GMM as described in greater detail below. For example, optionally, the model is constrained so that the body parts of the user are constrained in terms of the possible angles that they may assume. At 336, the mapped data is optionally integrated with video data.

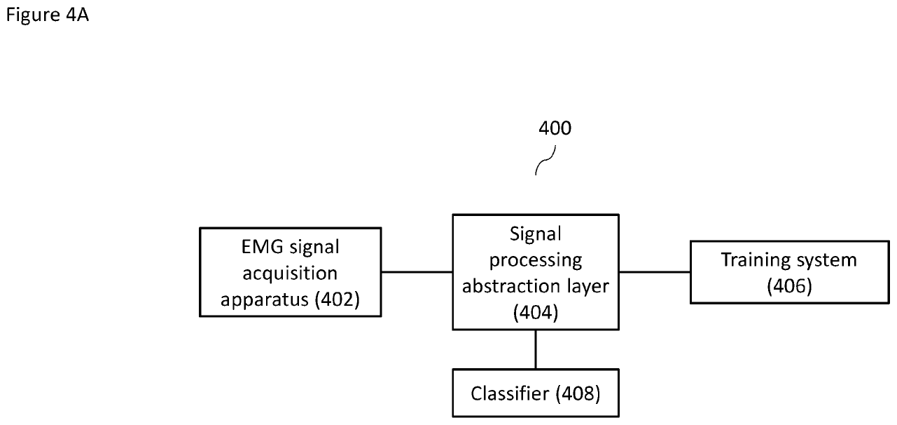

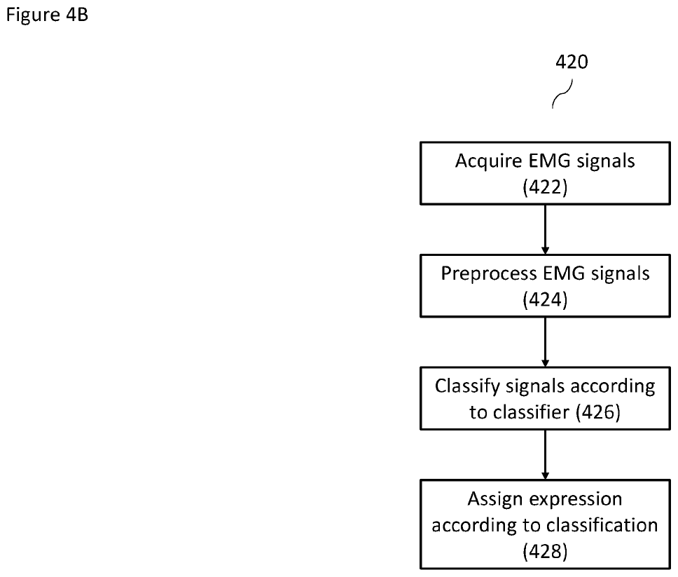

FIG. 4A illustrates an exemplary system for acquiring and analyzing EMG signals, according to at least some embodiments. As shown, a system 400 includes an EMG signal acquisition apparatus 402 for acquiring EMG signals from a user. In some implementations, the EMG signals can be acquired through electrodes (not shown) placed on the surface of the user, such as on the skin of the user (not shown). In some implementations, such signals are acquired non-invasively (i.e., without placing sensors and/or the like within the user). Optionally, at least a portion of EMG signal acquisition apparatus 402 may be adapted for being placed on a body part of the user, such as the face of the user as a non-limiting example. For such embodiments, at least the upper portion of the face of the user can be contacted by the electrodes. A non-limiting example of such an embodiment is disclosed in PCT Publication No. 2018/142228, published on 9 Aug. 2018, and owned in common with the present application, which is hereby incorporated by reference as if fully set forth herein.

EMG signals generated by the electrodes can then be processed by a signal processing abstraction layer 404 that can prepare the EMG signals for further analysis. Signal processing abstraction layer 404 can be implemented by a computational device (not shown). In some implementations, signal processing abstraction layer 404 can reduce or remove noise from the EMG signals, and/or can perform normalization and/or other processing in the EMG signals to increase the efficiency of EMG signal analysis. The processed EMG signals are also referred to herein as “EMG signal information.”

The processed EMG signals can then be classified by a classifier 408, e.g., according to the underlying muscle activity. In a non-limiting example, the underlying muscle activity can correspond to different facial expressions being made by the user. Other non-limiting examples of classification for the underlying muscle activity can include determining a range of capabilities for the underlying muscles of a user, where capabilities may not correspond to actual expressions being made at a time by the user. Determination of such a range may be used, for example, to determine whether a user is within a normal range of muscle capabilities or whether the user has a deficit in one or more muscle capabilities. As one of skill in the art will appreciate, a deficit in muscle capability is not necessarily due to damage to the muscles involved but may be due to damage in any part of the physiological system required for muscles to be moved in coordination, including but not limited to, central or peripheral nervous system damage, or a combination thereof.

As a non-limiting example, a user can have a medical condition, such as a stroke or other type of brain injury. After a brain injury, the user may not be capable of a full range of muscle movements or may not be able to fully execute certain muscle movements. As a non-limiting example, the user may have difficulty with one or more facial expressions, and/or may not be capable of fully executing a facial expression. As non-limiting example, after having a stroke in which one hemisphere of the brain experiences more damage, the user may have a lopsided or crooked smile. Classifier 408 can use the processed EMG signals to determine that the user's muscle movements are abnormal, such as for example that the user's smile is abnormal, and to further determine the nature of the abnormality (i.e., that the user is performing a lopsided smile) so as to classify the EMG signals even when the user is not performing a muscle activity in an expected manner.

As described in greater detail below, classifier 408 can operate according to a number of different classification protocols, such as: categorization classifiers; discriminant analysis (including but not limited to LDA (linear discriminant analysis), QDA (quadratic discriminant analysis) and variations thereof such as sQDA (time series quadratic discriminant analysis), and/or similar protocols); Riemannian geometry; any type of linear classifier; Naïve Bayes Classifier (including but not limited to Bayesian Network classifier); k-nearest neighbor classifier; RBF (radial basis function) classifier; neural network and/or machine learning classifiers including but not limited to Bagging classifier, SVM (support vector machine) classifier, NC (node classifier), NCS (neural classifier system), SCRLDA (Shrunken Centroid Regularized Linear Discriminate and Analysis), Random Forest; and/or some combination thereof.

The processed signals can also be used by a training system 406 for training classifier 408. Training system 406 can include a computational device (not shown) that implements and/or instantiates training software. For example, in some implementations, training system 406 can train classifier 408 before classifier 408 classifies an EMG signal. In other implementations, training system 406 can train classifier 408 while classifier 408 classifies muscle movements of the user, such as facial expressions, or a combination thereof. As described in greater detail below, training system 406, in some implementations, can train classifier 408 using known facial expressions and associated EMG signal information.

Training system 406 can also reduce the number of muscle movements for classifier 408 to be trained on, such as for example the number of facial expressions, for example to reduce the computational resources required for the operation of classifier 408 or for a particular purpose for the classification process and/or results. Training system 406 can fuse or combine a plurality of facial expressions in order to reduce their overall number. Training system 406 can also receive a predetermined set of facial expressions for training classifier 408 and can then optionally either train classifier 408 on the complete set or a sub-set thereof.

FIG. 4B shows an exemplary, non-limiting, illustrative method 420 for muscle movement classification according to at least some embodiments. As an example, at 422, a plurality of EMG signals can be acquired. In some implementations, the EMG signals are obtained as described in FIG. 4A, e.g., from electrodes receiving such signals from the muscles of a user, which may, for example and without limitation, comprise the facial muscles of a user. Other signals, such as image- or optical-based signals could be used including RGB or RGB-D (i.e., RGB and depth) optical signals.

At 424, the EMG signals can, in some implementations, be preprocessed to reduce or remove noise from the EMG signals. Preprocessing may also include normalization and/or other types of preprocessing to increase the efficiency and/or efficacy of the classification process. As one example, when using unipolar electrodes, the preprocessing can include reducing common mode interference or noise. Depending upon the type of electrodes used and their implementation, other types of preprocessing may be used in place of, or in addition to, common mode interference removal.

At 426, the preprocessed EMG signals can be classified using the previously described classifier. The classifier can classify the preprocessed EMG signals using a number of different classification protocols as discussed above with respect to FIG. 4A. Such classification may, for example, relate to any type of deviation from a normal set of signals, as determined from a plurality of users considered to be in the normal range for functionality of the muscles whose EMG signals are being obtained.

Examples of classification methods which may be implemented include, but are not limited to, Riemannian geometry and QDA or sQDA.

As a non-limiting example, the classification may relate to facial expressions. Facial expression classification may also be performed according to categorization or pattern matching, against a data set of a plurality of known facial expressions and their associated EMG signal information.

Turning back to stage 426, the classifier, in some implementations, can classify the preprocessed EMG signals to identify facial expressions being made by the user, and/or to otherwise classify the detected underlying muscle activity as described in the discussion of FIG. 4A. At 428, the classifier can, in some implementations, determine a facial expression of the user based on the classification made by the classifier.

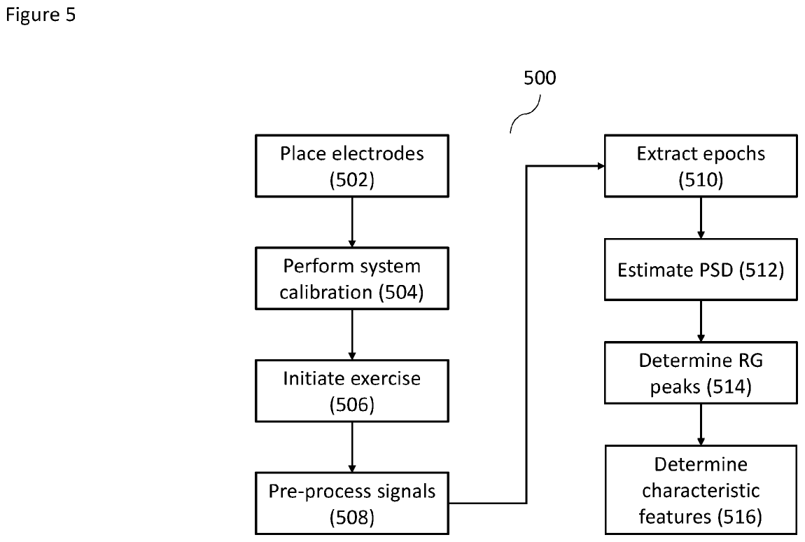

FIG. 5 shows a non-limiting, exemplary EEG flow. In a flow 500, the process preferably begins by placing the electrodes on the user in 502. Next, system calibration is performed at 504, at least for the electrodes to determine that they're working properly and that they have been calibrated for this session with the user. An exercise or other action is then preferably initiated in 506. The signals obtained from the EEG while the exercise is being performed are then preferably preprocessed in 508. One or more epochs may be extracted in 510 followed by estimation of PSD (2-D spectral representation of EEG data) at 512. Next the RG peaks are determined in 514. Characteristic features of the EEG may then be determined in 516.

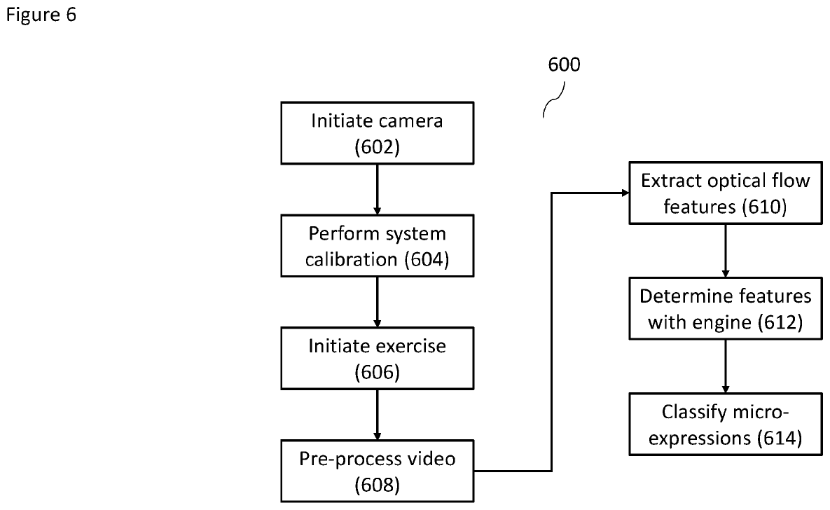

FIG. 6 describes a non-limiting, exemplary optical micro expression flow (also referred to as “optical flow” throughout). In a flow 600, the camera is initiated in 602, and is then calibrated optionally by system calibration in 604. These steps may be performed for example, as described regarding FIG. 1A. Next, an exercise or other action by the user is initiated in 606. As these performs the exercise or other action, video data is obtained and is preferably preprocessed in 608.

Optical flow features from video data are preferably extracted in 610. The features are determined with an engine 612 to, for example assemble them into an analysis and/or determine their importance or relative weight. The micro expressions of the users are preferably classified in 614, which may reveal a correlation to one or more emotions of the user, feelings of the user. Individual micro expressions represent a signal which may be correlated to underlying emotions when coherent to other biosignal modalities that are coherent to an underlying emotion. Optionally, a combination of such micro expressions with one or more biosignal modalities produce coherent correlates that provide a higher probability of accurately characterizing an underlying emotion. Such information also has diagnostic value because the micro expressions may be compared to those of a normal user that is a user not affected by a neural disease or injury.

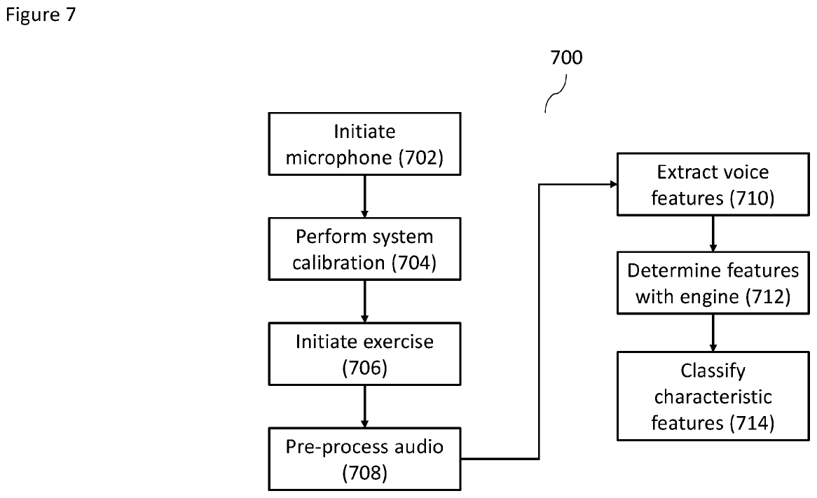

FIG. 7 describes an exemplary non-limiting flow for analyzing the voice of the user. In a flow 700, the microphone is initiated in 702, and is then calibrated in 704 preferably as part of the system calibration. As previously described, an exercise or other action by the user is initiated in 706. While the action or exercise is being performed by the user, preferably the audio of the user's voice that is obtained while this extra exercise is being performed is preprocessed in 708. One or more voice features are preferably extracted in 710. Such features may include but are not limited to phonemes and visemes. Such features are determined with engine 712, for example, to determine their weight or importance or other characteristics. Next, the characteristic features are properly classified in 714, for example, to determine stress, whether the user is able to speak normally and the like.

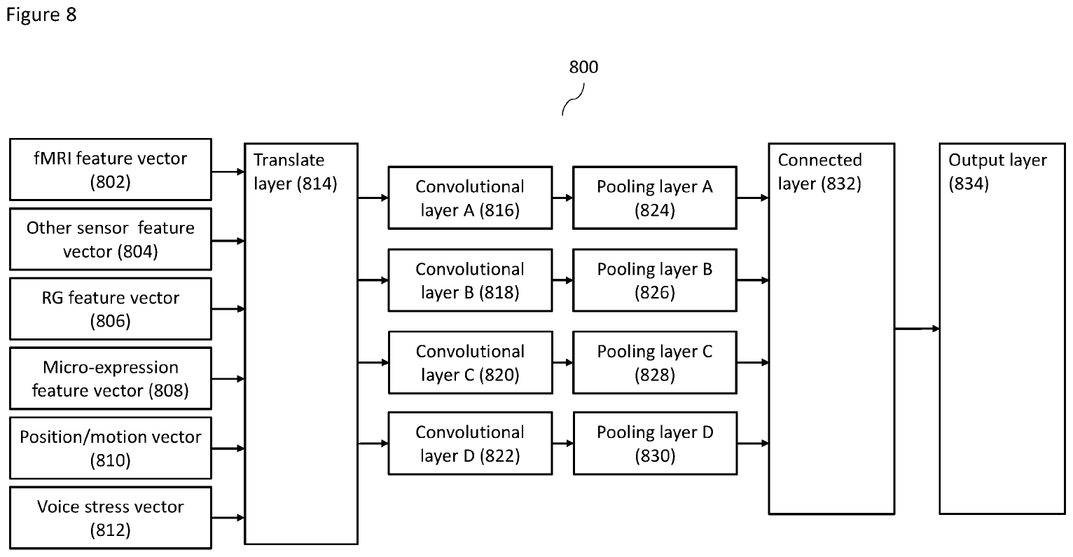

FIG. 8 relates to a non-limiting, exemplary convolutional and pooling neural network with combined connected layers as a non-limiting example of a neural network that may be used with the present invention. As shown in a system 800, preferably a plurality of vectors are fed into the neural net. These vectors include but are not limited to an fMRI feature vector 802, other sensor feature vector 804, an RG feature vector 806, a micro expression feature vector 808, a position motion vector 810, which preferably relates to tracking the position of the user and the voice stress factor 812.

All of these vectors are preferably fed into a translation layer 814 so they may be converted to an appropriate shared format or structure or at least transformed into a structure that allows them to be manipulated and considered by the neural net within a single context. As a non-limiting example and as described in greater detail below, such translation preferably relates to transforming the vectors into Hilbert space.

Next, the output of the translation 814 preferably includes a plurality of convolutional layers. 816, 818, 820 and 822 show convolutional layers A, B, C, and D, respectively. The convolutional layers analyze the information followed by pooling layers, A, B, C and D, or 824 to 830. The information is then fed to a single connected layer 832, which is then provided to a single output layer 834, thereby enabling different data types to be combined in a single analysis.

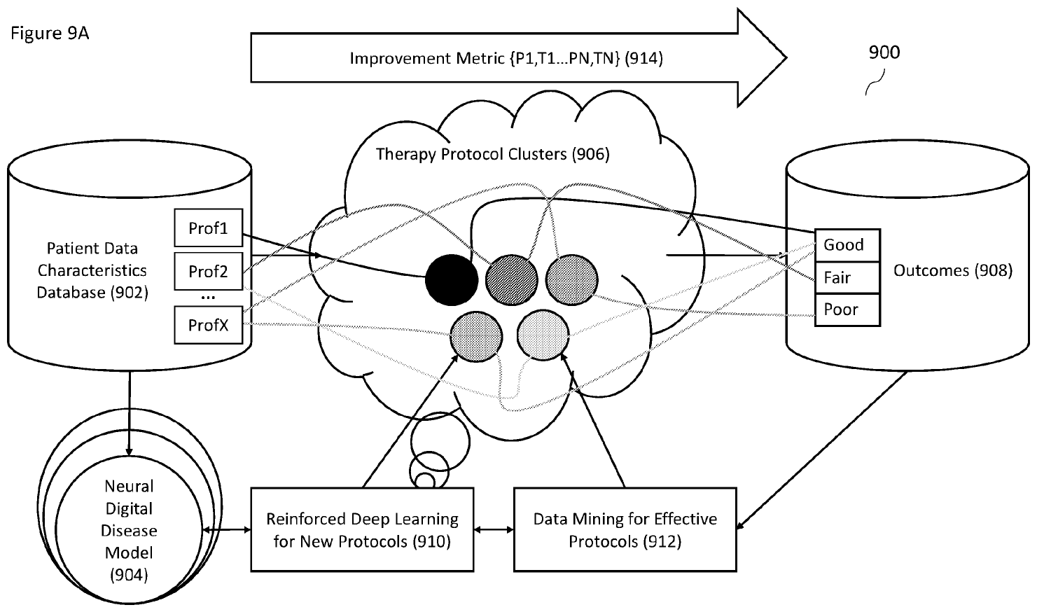

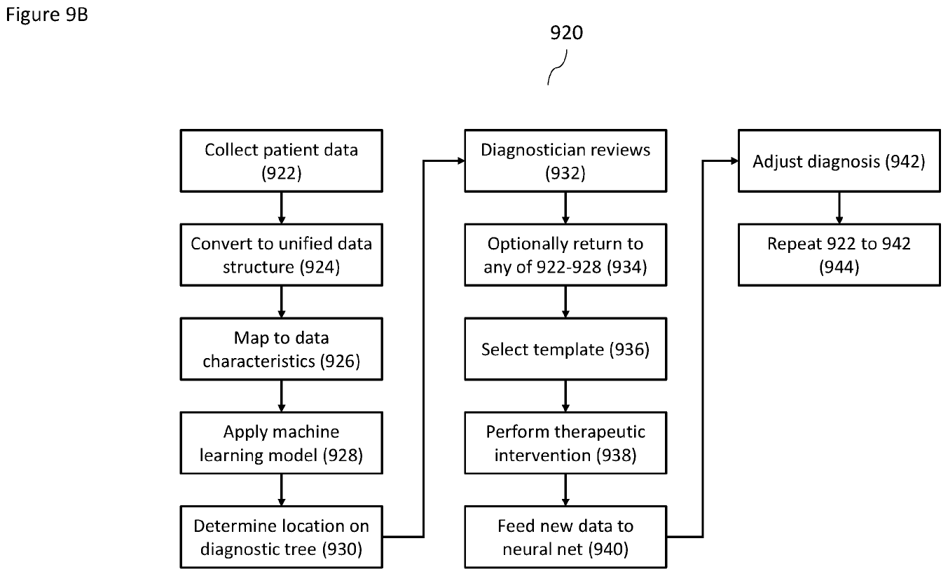

FIG. 9A relates to a non-limiting, exemplary flow for analysis and treatment. As shown in a flow 900, preferably a patient data characteristics database 902 includes a plurality of profiles which may be for example, Profs (profiles) one, two, and others and which may relate to different types of patients and/or different patient diagnoses. The patient data is then preferably fed into a neural digital disease model 904 which preferably relates to a plurality of different diseases, but optionally features a plurality of different models each relating to a single neural disease, injury or condition.

The information from the patient data characteristics database 902 is also preferably fed to therapy protocol clusters 906. These preferably relate to reinforce deep learning for new protocols 910, and data mining for effective protocols 912. The existing therapy protocol clusters 906 may then be updated according to the outcome shown in 908, and also optionally according to data mining and then reinforced deep learning.

FIG. 9B relates to a non-limiting, exemplary expanded feedback loop for diagnosis and optional therapeutic intervention. In a feedback loop flow 920, patient data is preferably collected in 922. The data is then converted to unified data structure in 924. The unified data structure information is preferably mapped to data characteristics 926, followed by applying a machine learning model in 928. Optionally, more than one machine learning model may be applied in 928 or alternatively a single model, but with a plurality of layers and/or other sub components such as that shown for example, in FIG. 8, may be applied to analyze a plurality of different data types.

After the application of machine learning mapped to the data, the location of the patient on a diagnostic tree in 930 is preferably determined. The diagnostic treatment options include a plurality of different branches and/or a plurality of different conditions. It is possible in many cases that the condition of a user may fall upon a spectrum and/or may include multiple different conditions or sub conditions. The diagnostic tree allows these complexities to be captured.

Preferably, a diagnostician reviews the location on the diagnostic tree at 932 and may optionally invoke a return of the process to any of steps 922 to 928 as appropriate in 934. Once the location on the tree has been determined, preferably a template for treatment is selected at 936 according to previously tested models for treatment on different patients. Then a therapeutic intervention is performed in 938. New data from the therapeutic intervention is preferably fed to the neural net in 940. Optionally, the diagnosis is adjusted at 942, preferably steps 922 to 942 are repeated in step 944.

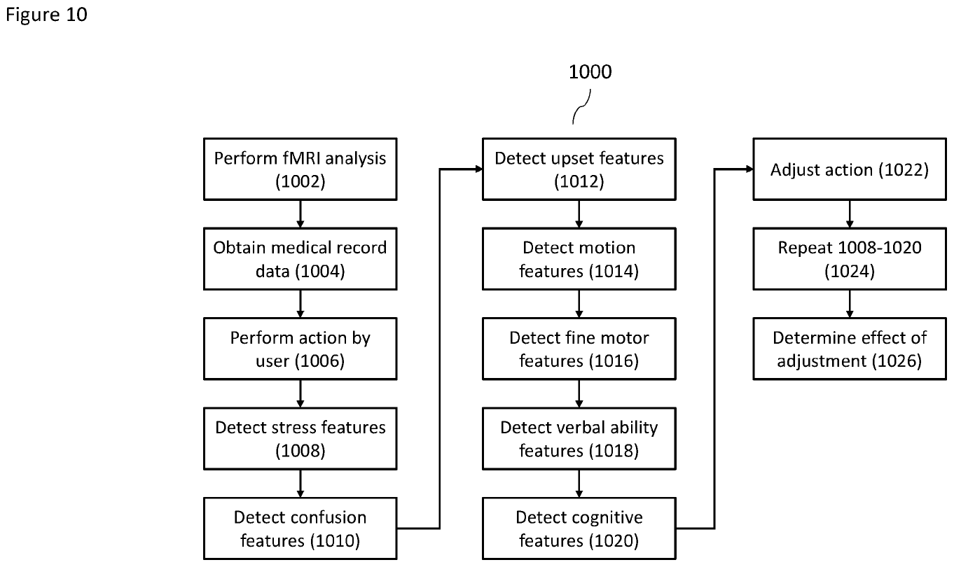

FIG. 10 relates to a non-limiting, exemplary neural disease diagnostic classification method. As shown in the method 1000, the method preferably begins with fMRI analysis of the patient's brain at 1002. Medical record data which may be structured or unstructured is preferably obtained in 1004 and is preferably converted to a format which is suitable for analysis with the other types of data. For example, by converting unstructured data to structured data by determining one or more features of the data that are important for further analysis. Those of skill in the art can appreciate that methods in accordance with FIG. 10 which use fMRI could be useful for some therapies but not others depending on the neurological condition.

Some neurological indications may benefit from frequent sampling of the progress to an outcome as predicted by a prognosis, while other conditions may be more reliant on an initial diagnostic testing. For some conditions, such as stroke, this sampling may be a complete sampling of the patient data (including imaging such as fMRI) as taken at the time of diagnosis. This initial sampling may give a complete and accurate sample of the patient progress. For determining the progress of stroke patients, while conducting radiological imaging for an initial diagnosis, it is not clear that additional imaging after the diagnosis is beneficial. While there is ongoing research into the benefits of therapy support with more frequent imaging, at this time there is little data to support this benefit for stroke patients (see, for example, Ward et al., “Neural correlates of motor recovery after stroke: a longitudinal fMRI study”, Brain. 2003 November; 126(0 11): 2476-2496). Given the cost to collect fMRI data and the radiation to which the patient is exposed, it is expected in the short to medium term (or such time as there is conclusive evidence of significant benefit) that in stroke therapy this will not be part of the feedback biosignal sampling. However, other neurological indications may benefit from additional sampling after the initial diagnosis.

Non-limiting examples of such additional neurological indications include dementia, epilepsy, headache disorders, multiple sclerosis, neuroinfection, neurological disorders associated with malnutrition, pain associated with neurological disorders, Parkinson's disease and traumatic brain injuries.

Preferably an action is performed by the user in 1006 and then one or more “stress-related” features are detected in 1008. Optionally, one or more “confusion” features that are detected in 1010, one or more upset features are detected in 1012, one or more motion features are detected in 1014, one or more fine motor features are detected in 1016, one or more verbal ability features are detected in 1018, and one or more cognitive features are detected in 1020. Optionally the process of detecting the features from 1008 to 1020 is performed simultaneously, although the process may be performed sequentially or in groups or pairs of actions. Next, preferably an action for the user to perform is suggested in 1022, for example, according to feedback from the system or from a therapist. Preferably steps 1008 to 1020 are repeated 1024, and the effect of the adjustment is determined in 1026.

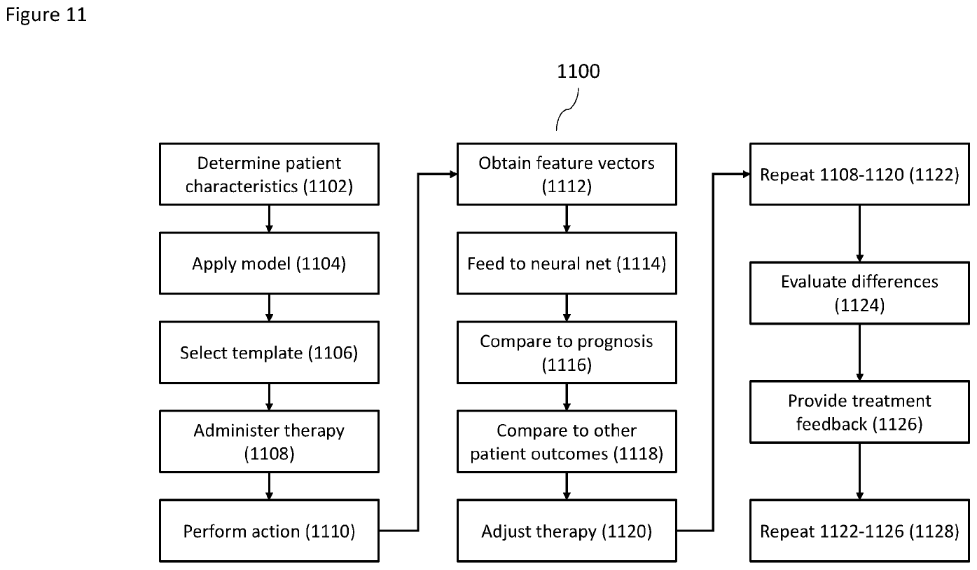

FIG. 11 relates to a non-limiting, exemplary analysis flow for treatment. As shown in the flow 1100, one or more patient characteristics are preferably determined in 1102. Such patient characteristics may relate to functionality of the patient according to one or more different types of modalities, including without limitation neurological function, muscle function, cognitive function and the like. Such functions may be measured according to any suitable modality, for example as described herein. Optionally, such characteristics relate to personality characteristics of the patient, in order to determine which types of therapy, including without limitation which types of therapeutic exercises, would be mostly useful.

Other non-limiting characteristics may include demographic information, motion analysis (including without limitation the ability of the patient to perform certain movements), the effect of any previously administered therapy and so forth.

Next, the model is applied and 1104 which is preferably a neural net or other machine learning model. The patient characteristics preferably include different types of patient data as previously described. Next a template for therapy is selected in 1106 and the therapy is administered in 1108. Then the patient performance and action are determined in 1110. Preferably one or more feature vectors are determined 1112 for example as described regarding FIG. 8.

The feature vectors are preferably fed to a neural net in 1114, and this output of this neural net is preferably then compared to a prognosis in 1116 such as for example, the user's or patient's previous prognosis, or prognosis according to probabilities between different patients. Preferably also the prognosis is compared to other different patient outcomes in 1118. The therapy is optionally adjusted in 1120 according to this comparison and then preferably steps 1108 to 1120 are repeated at least once in 1122. The differences in the user or patient state and/or one or more of the patient characteristics or features is preferably then determined in 1124. Treatment feedback is preferably provided in 1126 for example, to adjust the treatment. The treatment feedback maps are preferably provided to the therapist or directly to the patient. Next, stages 1122 to 1126 are preferably repeated at least once in 1128.

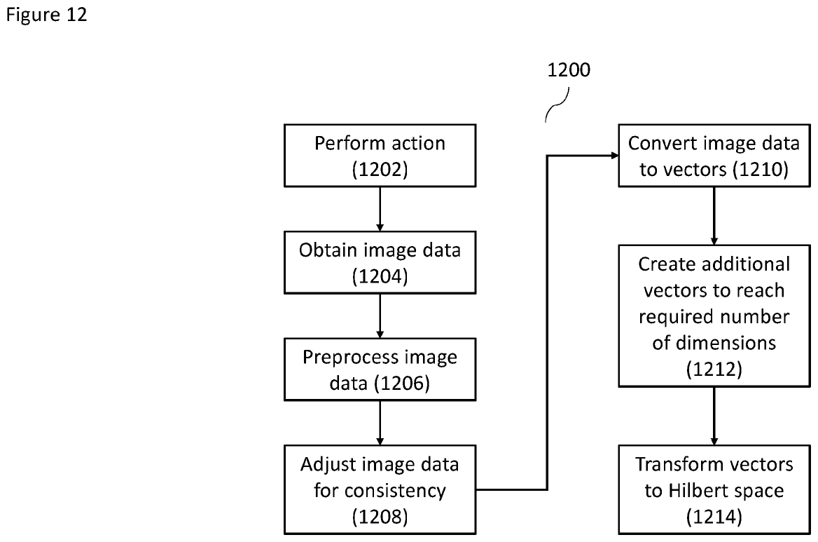

FIG. 12 relates to a non-limiting exemplary method for converting image data to a Hilbert space, by applying a Hilbert transform. As shown in a flow 1200, an action is performed by the user in 1202, and image data is obtained in 1204. For example, these actions may be performed with the system of FIG. 1A as previously described. The image data is preferably preprocessed in 1206, for example, for histogram brightness, for noise and for other types of processing which would assist in further analysis and transformation to the Hilbert space.

The image data is preferably adjusted for consistency in 1208. Such an adjustment may relate to errors or problems in one or more frames of such image data, including without limitation brightness issues, other artifacts which may need to be adjusted. The image data is then preferably converted to vectors in 1210. Such a conversion allows image data to be represented as a vector. Preferably additional vectors are created to reach the required number of dimensions in 1212 for the transformation of the vectors to Hilbert space. Then the vectors are transformed to Hilbert space in 1214 after the required number of dimensions in 1212 is determined according to whether the Hilbert spaces should be a shared Hilbert space between the plurality of different data types. Such a shared Hilbert space would not only be for image data, but also for EEG data, EKG data, fMRI data or other types of data.

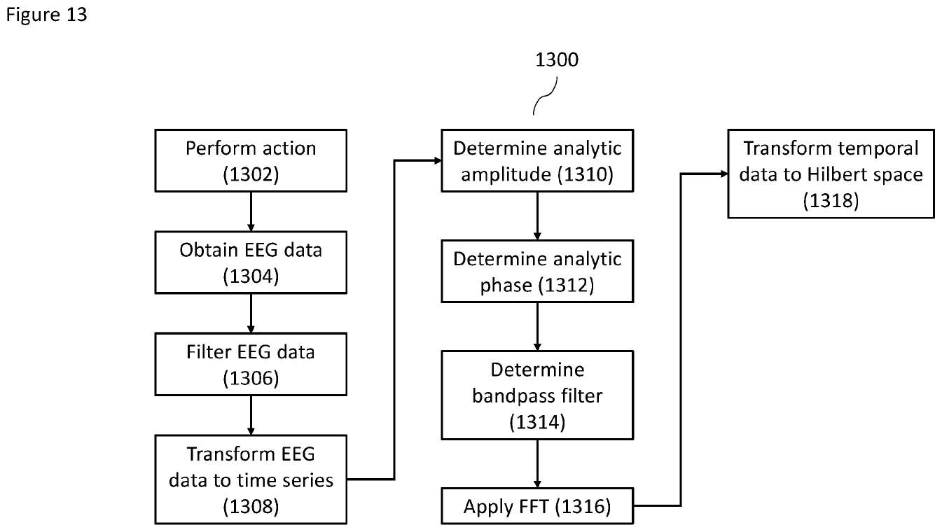

FIG. 13 relates to a non-limiting, exemplary method for transforming EEG data into the Hilbert space, by using the Hilbert transform. In a flow 1300, the user preferably performs an action as prescribed previously in 1302. Next, the EEG data is obtained in 1304 and is then filtered in 1306. The filtered EEG data is preferably transformed to a time series at 1308, which may be done in a plurality of different ways. A problem exists with multi-electrode biosignal data, which may for example be obtained with EEG and ECG where multiple electrodes are positioned on the body of the patient, in that multiple signals are spatially distributed, while the signals themselves change over time. To overcome this problem and detect events, optionally a pre-processing phase is performed, including segmentation of the biosignal data (e.g., to X second sequences) and selection of filters. The result is a transformed time series.

The analytic amplitude is preferably determined at 1310, followed by determining the analytic phase in 1312. After that, a bandpass filter is determined and applied in 1314. After that, the FFT (Fast Fourier Transform) may then be applied in 1316 and the temporal data is then transformed to the Hilbert space in 1318.

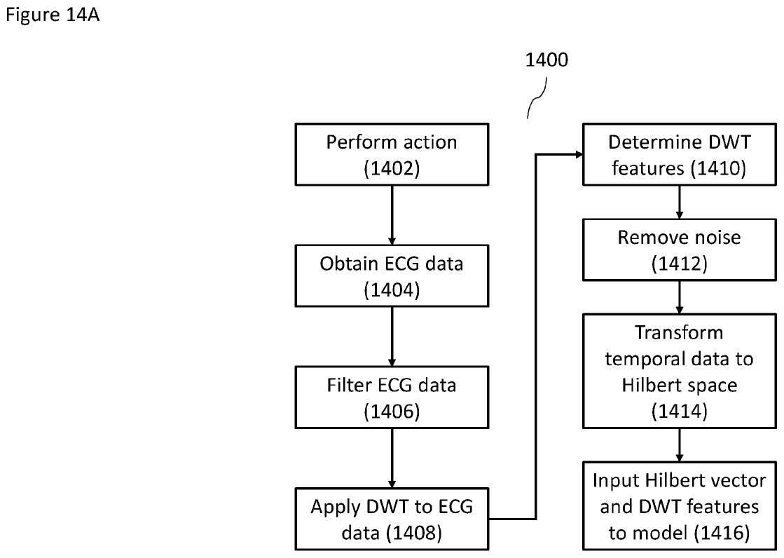

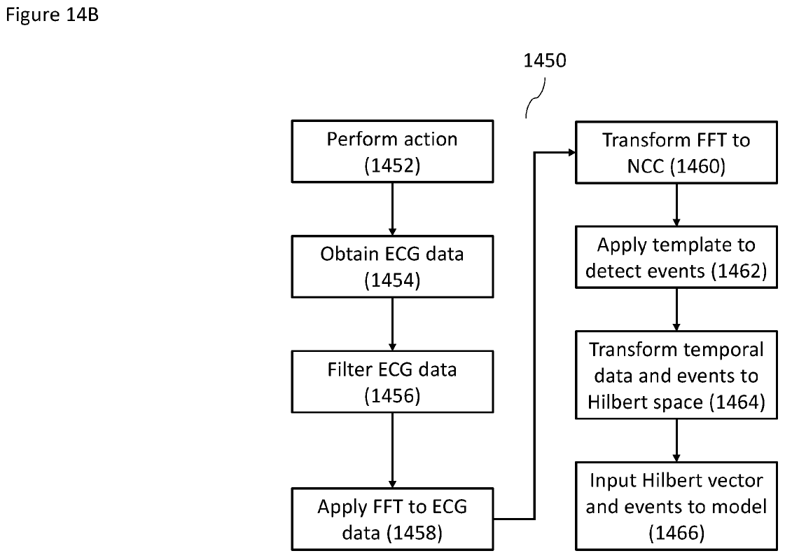

There are varied techniques to represent biosignal data into easy-to-use features, while simultaneously capturing transient changes in these features as biosignals traverse evoked events. Non-limiting examples of such techniques include DCC, DWT (Discrete wavelet Transform) and DFT. These techniques may be applied to the methods of FIGS. 13, 14A and 14B, for example.

FIGS. 14A and 14B relate to non-limiting, exemplary flows for applying the Hilbert space or the Hilbert transform to ECG data. Turning now to FIG. 14A, as shown in the flow 1400, the user again performs an action in 1402, and ECG data is obtained in 1404. The ECG data is filtered in 1406. Optionally, a DWT transform is applied in 1408. The DWT transform may be used in place of or in addition to an FFT, a short Fast Fourier Transform correlated to a time series analysis as described above, or other types of temporal transforms, as described for example with regard to FIG. 14B. Other types of wavelet transforms may be applied, as described for example in Nagendra et al., “Application of Wavelet Techniques in ECG Signal Processing: An Overview”, International Journal of Engineering Science and Technology (IJEST), Vol. 3 No. 10 Oct. 2011, pp 743FF). Preferably then DWT features are determined in 1410 and noises are removed in 1412. The denoised DWT signal is now denoised temporal data and is preferably transformed into Hilbert space in 1414. Optionally, the Hilbert vector and DWT features may be input to a model such as, for example, in a neural net, which may for example, be the previously described model of FIG. 8 and as shown in 1416.

FIG. 14B shows a similar flow to the method of FIG. 14A, except that a normalized cross-correlation (NCC) is computed by using an FFT, as described in “Computation of the normalized cross-correlation by fast Fourier transform” by A. Kaso (PLoS ONE 13(9): e0203434). The NCC may then be transformed to Hilbert space. As shown in the flow 1450, the user again performs an action in 1452, and ECG data is obtained in 1454. The ECG data is filtered in 1456. Next the FFT is applied to the ECG data in 1458. The FFT is then transformed to the NCC in 1460. A template is applied to the NCC-transformed data in 1462, in order to detect one or more events. The temporal data and the events are then converted to Hilbert space, for example as described above, in 1464. The Hilbert vector and the events are input to the model in 1466.

### Example—Diagnosis, Prognosis and Treatment of Illustrative Neurological Diseases

There are four aspects of this invention that relate to the diagnosis, prognosis and treatment (and/or therapy) that applies to disease types described herein, including without limitation dementia, epilepsy, multiple sclerosis, Parkinson's disease, stroke, amyotrophic lateral sclerosis (ALS), post-traumatic stress disorder (PTSD) and traumatic brain injuries.

Aspect 1—Diagnosis and Classification: It is important to correctly diagnose and classify the patient, this establishes the baseline or start point for subsequent prognosis stages, and the tracking of the patient progress.

Aspect 2—Prognosis: Having established the diagnosis and baseline, a prediction of patient progress to an outcome (after a given period and treatment/therapy) is made.

Aspect 3—Tracking of Treatment/Therapy: By the processes articulated in 1 and 2 above, a beginning point and end point of the predicted progress of the patient are established. The path between these points is a result of treatment/therapy and the natural progression of the neurological disease. Statistically for any given protocol of therapy/treatment and time we would normally expect a scatter plot of progression. Though individual patients will be expected to respond uniquely to the applied therapy/treatment for a variety of known/unknown reasons. As one aspect for the better understanding the effects of various possible treatments/therapy, optionally these plots are constructed in the context of statistical scatter plots.

Aspect 4—Discovery of Underlying Factors Not Present in Current State of the Art Prognosis: With the collection of significant baseline data at tune of Diagnosis and Classification, and the tracking of a significant cohort (dataset of patients tracking through different therapies), it is possible to determine whether some Therapies/Treatment perform better/worse for some specific patient characteristics, this will enable more appropriate/effective prognosis, and pivots during therapy/treatment of unique patient types. This process may also be termed a Cybernetic Feedback process.

### Parkinson's Disease

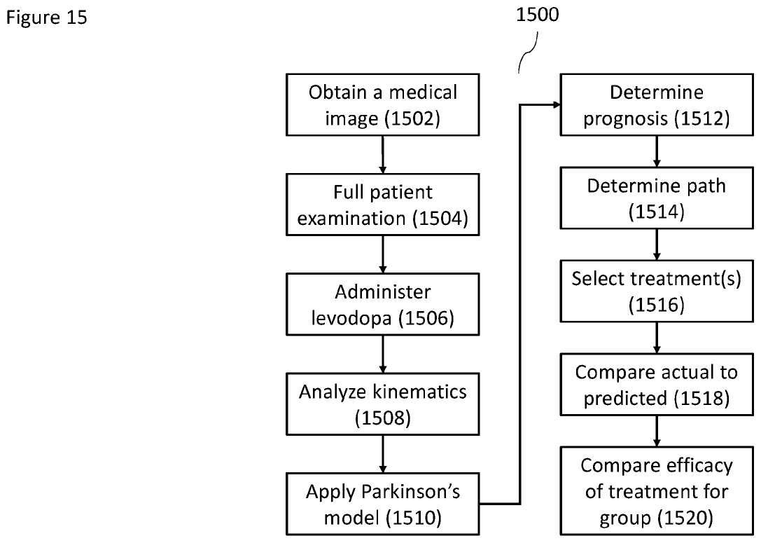

Turning now to the drawings, FIG. 15 relates to an exemplary process, using the above four general stages, for the treatment of a subject suffering from Parkinson's disease. These stages are described in greater detail below in a non-limiting, exemplary embodiment in relation to a process 1500.

### Diagnosis

No single specific test exists to diagnose Parkinson's disease. A diagnosis is based upon a subject's medical history, a review of signs and symptoms, and a neurological and physical examination, as shown with regard to stage 1504. Optionally, a specific single-photon emission computerized tomography (SPECT) scan called a dopamine transporter (DAT) scan is performed. Also, optionally, imaging tests, such as MRI, CT, ultrasound of the brain, and PET scans, are performed; but typically such tests are performed to help rule out other disorders. These various types of scans are included as stage 1502.

Another form of diagnosis involves the administration of carbidopa-levodopa (e.g., Rytary, Sinemet, others), a Parkinson's disease medication, in a sufficient dose to show the benefit, as low doses for a day or two aren't reliable. Significant improvement with this medication may confirm the diagnosis of Parkinson's disease. This is shown as stage 1506.

Sometimes it takes time to diagnose Parkinson's disease. Doctors may recommend regular follow-up appointments with neurologists trained in movement disorders to evaluate the subject's condition and symptoms over time and diagnose Parkinson's disease. For example kinematics may be analyzed as previously described and as shown in 1508.

### Prognosis

Having established the diagnosis and baseline, a prediction of patient progress to an outcome (after a given period and treatment/therapy) is made (based on best current models of Parkinson's).

Crucially, in the case of Parkinson's disease prognosis, current expectations are that it cannot be cured. However, life style changes, medications, surgery and non-invasive/invasive application of electro-magnetic stimulus (e.g., DBD TIN) can help control the symptoms, often dramatically mitigating them.

The present invention, in at least some embodiments, provides a model to record the diagnosis/classification baseline, as shown in 1510. The prognosis prediction of patient state to an outcome is then determined in 1512. In the case of Parkinson's, the objective datapoints collected should not be expected to result in improvements in the underlying disease, rather amelioration of the symptomatic impacts—these results would be recorded and plotted.

### Tracking of Treatment

A beginning and end point of the predicted progress of the patient are determined with regard to Parkinson's disease. The path between these points is a result of treatment/therapy and the natural progression of Parkinson's. The path is preferably determined in stage 1514. Statistically for any given protocol of therapy/treatment, and time, one would normally expect a scatter plot of progression. However, individual patients will be expected to respond uniquely to the applied therapy/treatment for a variety of, as yet, known/unknown reasons. As a useful step to the better understanding, the effects of various possible treatments/therapy this invention provides a mechanism to construct these unique patient progression plots in the context of a generic cohort statistical scatter plot.

Treatment(s) are preferably selected in stage 1516. Medications may help a patient to manage problems with walking, movement, and tremor. These medications increase or substitute for dopamine, for example including but not limited to Carbidopa-levodopa. Medical treatments can include, but are not limited to the following:
Levodopa, the most effective Parkinson's disease medication, is a natural chemical that passes into your brain and is converted to dopamine. Levodopa is combined with carbidopa (Lodosyn), which protects levodopa from early conversion to dopamine outside your brain. This prevents or lessens side effects such as nausea.
Carbidopa-levodopa infusion. Duopa is a brand-name medication made up of carbidopa and levodopa. However, its administered through a feeding tube that delivers the medication in a gel form directly to the small intestine.
Dopamine agonists. Unlike levodopa, dopamine agonists don't change into dopamine. Instead, they mimic dopamine effects in the brain. Dopamine agonists include pramipexole (Mirapex), ropinirole (Requip) and rotigotine (Neupro, given as a patch). Apomorphine (Apokyn), is a short-acting injectable dopamine agonist used for quick relief.
MAO B inhibitors. These medications include selegiline (Eldepryl, Zelapar), rasagiline (Azilect) and safinamide (Xadago). They help prevent the breakdown of brain dopamine by inhibiting the brain enzyme monoamine oxidase B (MAO B). This enzyme metabolizes brain dopamine.
Catechol O-methyltransferase (COMT) inhibitors. Entacapone (Comtan) is the primary medication from this class. This medication mildly prolongs the effect of levodopa therapy by blocking an enzyme that breaks down dopamine.
Tolcapone (Tasmar) is another COMT inhibitor that is rarely prescribed due to a risk of serious liver damage and liver failure.
Anticholinergics. These medications were used for many years to help control the tremor associated with Parkinson's disease. Several anticholinergic medications are available, including benztropine (Cogentin) or trihexyphenidyl.
Amantadine. Doctors may prescribe amantadine alone to provide short-term relief of symptoms of mild, early-stage Parkinson's disease. It may also be given with carbidopa-levodopa therapy during the later stages of Parkinson's disease to control involuntary movements (dyskinesia) induced by carbidopa-levodopa.

### Surgical Procedures

Deep brain stimulation. In deep brain stimulation (DBS), surgeons implant electrodes into a specific part of the brain. The electrodes are connected to a generator implanted in the chest near the collarbone that sends electrical pulses to the brain to reduce Parkinson's disease symptoms.

Deep brain stimulation is most often offered to people with advanced Parkinson's disease who have unstable medication (levodopa) responses. DBS can stabilize medication fluctuations, reduce or halt involuntary movements (dyskinesia), reduce tremor, reduce rigidity, and improve slowing of movement.

DBS is effective in controlling erratic and fluctuating responses to levodopa or for controlling dyskinesia that doesn't improve with medication adjustments.

The exemplary methods of the present invention as described herein provides a means to check the actual realization of a cohort of patients who receive varied treatment and therapy, effectively providing clusters of scatter plots that help to validate that the Prognosis prediction is vindicated or in need of refinement, shown as stage 1518. Furthermore, it is possible to adjust the DBS treatment parameters according to the previous experiences of patients having a certain prognosis.

### Discovery of Underlying Factors not Present in Current State of Art Prognosis

With the collection of significant baseline data at time of Diagnosis and Classification, and the tracking of a significant generic cohort (dataset of patients tracking through different therapies) it is possible to determine some Therapies/Treatment perform better/worse for some specific patient characteristics, in a predictive manner. If so, this will enable more appropriate/effective prognosis, and pivots during therapy/treatment of unique patient types.

The present invention, in at least some embodiments, provides a means to recognize anomalies in actual versus predicted performance and support changes in Treatment/Therapy to ameliorate specific protocols to effect optimum personalization, shown as stage 1520.

### Amyotrophic Lateral Sclerosis

A generically similar process to the above can be performed for ALS (amyotrophic lateral sclerosis), which is largely a process of diagnosis through elimination. The above process can be adapted for ALS diagnosis and treatment, including with regard to treatment of the symptoms.

### Stroke

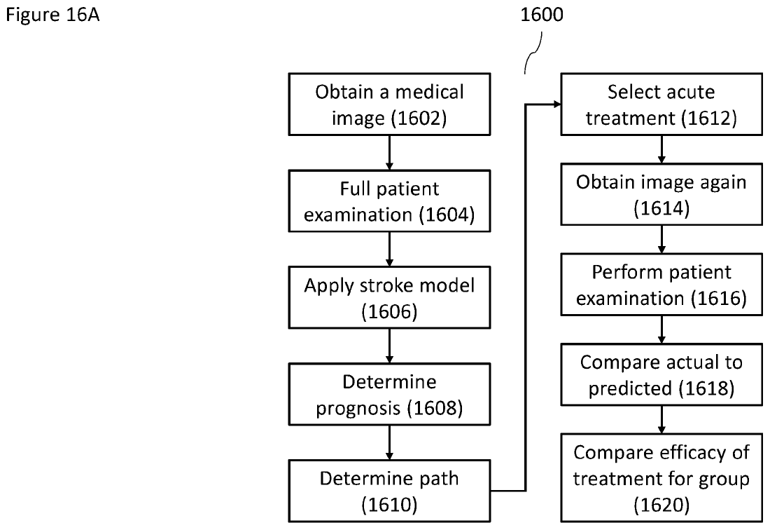

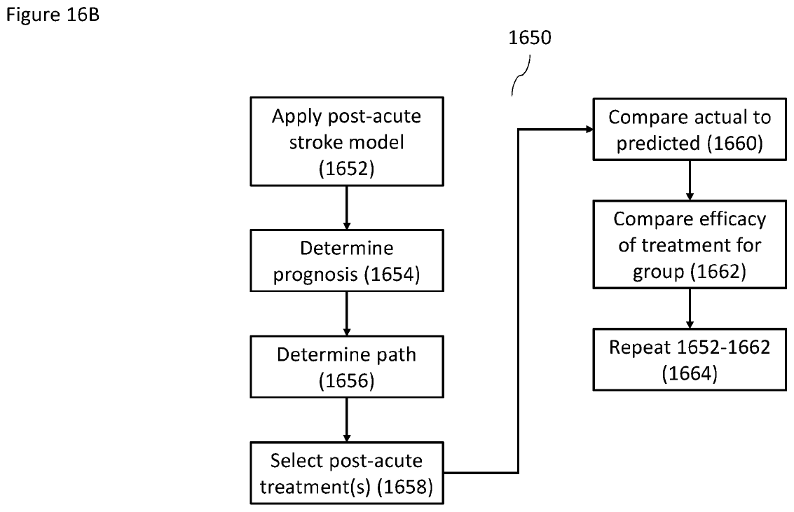

FIGS. 16A and 16B relate to exemplary processes, using the above four general stages, for the treatment of a subject suffering from a stroke. These stages are described in greater detail below in a non-limiting, exemplary embodiment in relation to a process for acute stroke treatment in FIG. 16A and for post-acute stroke treatment in FIG. 16B.

FIG. 16A shows a non-limiting, exemplary process 1600 for treatment of acute stroke.

### Diagnosis

Diagnosis of a stroke preferably includes one or more imaging tests such as MRI, CT, cerebral angiogram, ultrasound of the brain and/or carotid, and PET scans, as shown in stage 1602. More preferably CT, cerebral angiogram and/or fMRI is performed. Optionally a subject's medical history, a review of signs and symptoms, and a neurological and physical examination are also considered, as shown with regard to stage 1604. Affected functions that may be detected include but are not limited to plegia (any type of paralysis), balance, aphasia (speech related difficulties), apraxia (difficulty with any type of skilled movement), attention, executive functions (for example, judgement), neglect and agnosia (inability to process sensory information, including the loss of ability to recognize objects, persons, sounds, shapes, or smells).

### Prognosis

Having established the diagnosis and baseline, a prediction of patient progress to an outcome (after a given period and treatment/therapy) is made (based on best current models of stroke).

The present invention, in at least some embodiments, provides a model to record the diagnosis/classification baseline for stroke, as shown in 1606. The prognosis prediction of patient state to an outcome is then determined in 1608. For acute treatment, the underlying brain injury may be ameliorated. Therefore, prognosis may be repeated again in stage 1614 after acute treatment. Prognosis may be determined for example as proportional recovery of an affected limb. For example, it is known that for a majority of patients, within 6 months an affected limb will have recovered 70% of the total possible recovered function, but that this recovery depends upon corticomotor integrity and measurable corticomotor factors (ANN NEUROL 2015; 78:848-859, Byblow et al., “Proportional Recovery After Stroke Depends on Corticomotor Integrity”).

Other factors relate to the total lesion coverage in the brain and spinal cord, which may be viewed through imaging and which may be used to predict motor recovery (“Corticospinal Tract Lesion Load—A Potential Imaging Biomarker for Stroke Motor Outcomes”, Feng et al., Ann Neurol. 2015 December; 78(6): 860-870). Similarly, improvement in aphasia may also be predicted according to the initial severity of the lesions and their effect on speech (Lazar et al., “Improvement in Aphasia Scores After Stroke Is Well Predicted by Initial Severity”, Stroke. 2010; 41:1485-1488).

Optionally, diagnosis and prognosis are combined through a protocol such as PREP2, which has three steps. The first step of PREP2 is to establish a patient's SAFE score within 72 hours of stroke. The SAFE score is calculated by scoring Shoulder Abduction and Finger Extension separately, using the Medical Research Council grades. The patient's strength in each of these movements is scored between 0 and 5, where 0 is no muscle activity and 5 is normal strength and range of movement.

The second step of PREP2 involves the function of motor pathways between the stroke-affected side of the brain and the affected hand and arm using TMS (Transcranial Magnetic Stimulation). The TMS assessment is carried out 3-7 days after stroke. If TMS produces detectable responses (motor evoked potentials, MEP+), then the patient has potential for a Good functional outcome for the upper limb. If TMS does not produce detectable responses (MEP−), then the third step of PREP2 is needed.

The third step of PREP2 uses the patient's NIHSS score (National Institutes of Health Stroke Scale). This score is obtained 3 days after stroke for all patients with a SAFE score <5, in case they turn out to be MEP− and proceed to this final step. The NIHSS provides a measure of stroke severity, with higher scores indicating greater stroke severity. If the patient's NIHSS score is <7, they are most likely to have a Limited functional outcome for the upper limb. If the patient's NIHSS score is ≥7, they are most likely to have a Poor functional outcome for the upper limb.

At 1610, a treatment path is selected, based upon the diagnostic state of the patient, particularly with regard to whether the patient is suffering from an ischemic or hemorrhagic stroke. A beginning and end point of the predicted progress of the patient are determined with regard to stroke. The path between these points is a result of treatment/therapy and the natural progression of brain injury resulting from stroke. Statistically for any given protocol of therapy/treatment, and time, one would normally expect a scatter plot of progression. However, individual patients will be expected to respond uniquely to the applied therapy/treatment for a variety of, as yet, known/unknown reasons. As a useful step to the better understanding, the effects of various possible treatments/therapy this invention provides a mechanism to construct these unique patient progression plots in the context of a generic cohort statistical scatter plot.

### Tracking of Treatment

Acute treatment(s) are preferably selected in stage 1612, according to the type of stroke—ischemic or hemorrhagic stroke. An ischemic stroke relates to a clot blocking blood flow to the brain, which must be quickly treated to avoid or reduce damage. For example, drugs may be administered to dissolve clots if given within 4.5 hours of the stroke. Non-limiting examples of such drugs include intravenous injection of tissue plasminogen activator (tPA). Additionally or alternatively, emergency endovascular procedures may be performed directly inside the blocked blood vessel. Non-limiting examples of such procedures include medications delivered directly to the brain, removing the clot with a stent retriever.

Hemorrhagic stroke relates to bleeding from a blood vessel into the brain. Emergency treatment of hemorrhagic stroke focuses on controlling such bleeding and reducing pressure in the brain. Drugs may be given to lower blood pressure, reverse the effect of drugs that prevent blood clots and so forth. Optionally, surgery is performed to repair the source of bleeding, such as a blood vessel for example. The procedure may be performed for example if an aneurysm or arteriovenous malformation (AVM) or other type of vascular malformation caused the hemorrhagic stroke. These procedures include but are not limited to surgical clipping, to clamp the base of the aneurysm, to stop blood flow to it; coiling (endovascular embolization), to fill the aneurysm, which blocks blood flow into the aneurysm and causes the blood to clot; surgical AVM removal; stereotactic radiosurgery, by using multiple beams of highly focused radiation, stereotactic radiosurgery is an advanced minimally invasive treatment used to repair vascular malformations.

Optionally at stage 1614, imaging is performed again, to determine whether any injuries or structural risks in the brain have been ameliorated.

In stage 1616, the patient is examined to determine a response to the acute treatment and also to determine whether the acute stage has passed. Once the acute stage of the stroke has passed, the process may continue for the patient in the flow of FIG. 16B, for post-acute stroke.

In relation to assessing the actual response of the patient in comparison to what was predicted, preferably such an analysis is performed in stage 1618, by comparing the patient's results from the examination in stage 1616 to the prognosis in stage 1608. The relative response of the patient is preferably determined at this stage, to analyze in which respects the patient's results were better than expected, worse than expected or as expected. In stage 1620, the efficacy of treatment is compared overall for the group of patients receiving it. Such overall comparison may be used to update the prognostic models and patient profiles, for example.

Turning now to FIG. 16B, there is shown a non-limiting exemplary process for treatment of post-acute stroke. As shown in a process 1650, the process begins with applying a post-acute stroke model 1652. The post-acute stroke model relates to features of stroke after the initial acute phase, which typically lasts up to a few weeks. The post-acute phase may be further divided into two further phases, a second phase and a third phase. The second phase is the nonacute phase, and this may continue for a few months (typically up to six months) after the stroke. Finally, the chronic phase begins months to years after stroke. For the purpose of the below description, the nonacute and chronic phases may be combined, although optionally separate processes are developed for each such phase. During both the nonacute and chronic stroke phases, the patient may continue to recover lost functionality.

After application of the post-acute stroke model, the prognosis of the patient is determined in 1654. In the case of stroke, for post-acute treatment, the objective datapoints collected should not be expected to result in improvements in the underlying disease, rather amelioration of the symptomatic impacts—these results would be recorded and plotted.

At 1656, a treatment path is selected, based upon the diagnostic state of the patient, particularly with regard to whether the patient is suffering from an ischemic or hemorrhagic stroke. A beginning and end point of the predicted progress of the patient are determined with regard to stroke. The path between these points is a result of treatment/therapy and the natural progression of brain injury resulting from stroke in the post-acute phase. Preferably the statistically determined reactions of previous patients are used as a guide to determine future therapies. As a useful step to the better understanding, the effects of various possible treatments/therapy this invention provides a mechanist)) to construct these unique patient progression plots in the context of a generic cohort statistical scatter plot.

On the basis of the selected path, one or more treatments for the patient are selected at 1658. For example, these treatments may include physical and/or cognitive therapy. The actual response of the patient to these treatments is then compared to the predicted response at 1660. Stages 1658 and 1660 may be performed a plurality of times over a period of elapsed time.

At least once, and optionally periodically, the efficacy of the treatment for the patient is compared to that of the group in 1662. Stages 1652-1662 may be repeated at least once in 1664.

As a non-limiting example of a case study, a hypothetical patient, Maria, 72 years is considered. She is in the acute phase as she had an ischemic stroke on the right brain side very recently. She arrived at the stroke unit with symptoms of moderate-to-severe hemiplegia, hand spasticity, and signs of hemispatial neglect on the far space. She lives in a rural area, where out-patient rehabilitation settings may not be available.

The Clinical Decision Support System (CDSS) based on an international predictive modelling database, and implanted in the stroke unit and associated rehabilitation centers, suggests to her rehab clinician a personalized protocol that includes higher rehab dose. The rehabilitation dose may be delivered manually or through device mediated therapy. Non-limiting examples of device mediated therapy include neuromuscular electrical stimulation (NMES), specifically functional electrical stimulation (FES) that compensates for voluntary motion, and therapeutic electrical stimulation (TES) aimed at muscle strengthening and recovery from paralysis, and/or robotic arm support. In any case, such therapy is only provided once the patient is stabilized and able to receive therapy. Such a protocol may be performed during this acute phase for two weeks for example.

After 10 days for example, she is then transferred to the rehab clinic where she will join a group therapy 3× week with tailored robotic therapy in addition to daily PT and OT, and neuropsychological sessions with iVR (interactive voice response, optionally in a VR (virtual reality) setting) for hemispatial attention training for 3 months, which she will take further home. Such tailored robotic therapy may, for example, be performed with the MindMotion Pro product of MindMaze SA, Lausanne, Switzerland.

If the patient progresses well with the intensive and multimodal intervention, she may be discharged earlier than expected (after only 2 months, instead of 3). At home she will follow a semi-supervised and telemonitored physical therapy, in addition to the iVR spatial attention program, with monthly check visits to hospital, reducing burden of out-patient clinic. She may use for example a robotic therapy product, such as the MindMotion Go product of MindMaze SA, Lausanne, Switzerland.

### Non-Stroke Brain Injury

A similar process to the above may be performed for other types of brain injuries, such as traumatic brain injuries for example. Similar to stroke, scans such as CT and MRI may be helpful. Additionally, an intracranial pressure monitor may be inserted to determine the pressure within the skull, so that any swelling may be treated. Surgery may also be required.

Similarly to stroke, traumatic brain injuries have an acute phase and a post-acute phase. Treatments such as surgery are needed for the acute phase. During the post-acute phase, improvement may be seen upon application of various types of rehabilitative therapies, including therapeutic devices such as MindMotion Pro (MindMaze SA, Lausanne, Switzerland).

For both the acute and post-acute phases, the prognosis and treatment path may be determined as described above, but with adaptations to this specific patient population.

### Multiple Sclerosis

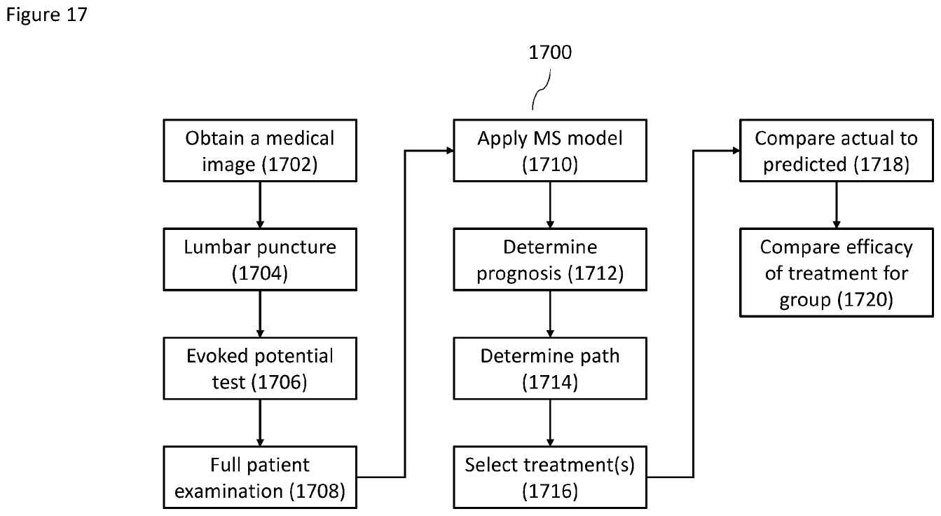

FIG. 17 relates to an exemplary process, using the above four general stages, for the treatment of a subject suffering from multiple sclerosis (MS). These stages are described in greater detail below in a non-limiting, exemplary embodiment for a process 1700.

### Diagnosis

The diagnostic stages are shown as stages 1702-1708. In stage 1702, an image of the patient's brain is obtained. Preferably an MRI is performed, which can reveal areas of MS (lesions) on the brain and spinal cord. The imaging process, such as the MRI, may be performed in conjunction with an intravenous injection of a contrast material to highlight lesions that indicate that the disease is in an active phase.

In stage 1704, a lumbar puncture is performed. In this process, fluid is withdrawn from the thecal sac that surrounds the spinal cord, and is tested for the presence of markers related to multiple sclerosis, such as specific antibodies for example. Optionally a blood test is performed to look for such markers, additionally or alternatively.

In stage 1706, an evoked potentials test is performed, to record the electrical signals produced by the nervous system in response to stimuli. An evoked potential test may use visual stimuli or electrical stimuli. For visual stimuli, the patient watches a moving visual pattern. Electrical stimuli may include short electrical impulses that are applied to nerves in the legs or arms. Electrodes measure how quickly the information travels down the nerve pathways.

In stage 1708, preferably a full patient examination is performed, optionally combining the results from one or more of the above tests and also considering diagnostic results that may or may not indicate MS. Preferably the examination includes kinematic movement analysis of the patient.

### Prognosis

After the diagnostic tests are performed, a model of MS is applied in 1710, to determine how the patient fits within various profiles of the disease. Next the prognosis for MS is determined in 1712. The prognosis preferably includes whether the patient is suffering from relapsing-remitting or primary-progressive MS. The prognosis also preferably includes a determination of whether the patient is suffering from an acute attack or the chronic form of the disease, as acute attacks may occur within the chronic form of the disease.

At 1714, a treatment path is selected, based upon the diagnostic state of the patient. A beginning and end point of the predicted progress of the patient are determined with regard to MS. The path between these points is a result of treatment/therapy and the natural progression of the disease. Statistically for any given protocol of therapy/treatment, and time, one would normally expect a scatter plot of progression. However, individual patients will be expected to respond uniquely to the applied therapy/treatment for a variety of, as yet, known/unknown reasons. As a useful step to the better understanding, the effects of various possible treatments/therapy this invention provides a mechanism to construct these unique patient progression plots in the context of a generic cohort statistical scatter plot. The treatment path is preferably focused on speeding recovery from attacks, slowing the progression of the disease and managing MS symptoms.

### Tracking of Treatment

One or more treatments are selected in 1716, according to the previously determined treatment path. A plurality of treatments may be selected, depending upon whether the patient is experiencing an acute attack or whether the disease is in a chronic state. MS is typically relapsing-remitting, meaning that sometimes patients experience acute attacks, while other times the disease is stable, with reduced symptoms.

For an acute attack, treatments may include: corticosteroids, such as oral prednisone and intravenous methylprednisolone, to reduce nerve inflammation; and (particularly if the patient doesn't respond to corticosteroids), plasma exchange (plasmapheresis).

For chronic management of the disease, the treatment depends upon the type of MS that is diagnosed. For relapsing-remitting MS, treatments include ocrelizumab, beta interferons, glatiramer acetate, dimethyl futnarate, fingolitnod, teriflunomide, natalizumab, alemtuzumab, and mitoxantrone.

For primary-progressive MS, currently the treatment is ocrelizumab, which slows progression of the disease.

### Discovery of Underlying Factors not Present in Current State of the Art Prognosis

In stage 1718, the actual response of the patient is compared to the predicted response. In stage 1720, the patient's response is compared to efficacy for the group. With the collection of significant baseline data at time of Diagnosis and Classification, and the tracking of a significant generic cohort (dataset of patients tracking through different therapies), it is possible to determine whether some Therapies/Treatment perform better/worse for some specific patient characteristics, in a predictive manner. If so, this will enable more appropriate/effective prognosis, and pivots during therapy/treatment of unique patient types. This is effectively a Cybernetic Feedback process.

With regard to MS, this method provides a means to recognize anomalies in actual versus predicted performance and support changes in Treatment/Therapy to ameliorate specific protocols to effect optimum personalization.

### Epilepsy

A similar process to the above for multiple sclerosis may be performed for epilepsy, with necessary adaptations to the specific patient population. For epilepsy, a different set of diagnostic tools may be employed, including an electroencephalogram (EEG) and/or a high-density EEG. Imaging modalities such as computerized tomography (CT), magnetic resonance imaging (MRI), functional MRI (fMRI), positron emission tomography (PET), single-photon emission computerized tomography (SPECT). Localization of the lesions can assist with treatment.

Treatment involves medication, surgery to ablate the area causing the surgeries and/or a vagal nerve stimulator, to act as a pacemaker for the brain.

The above process for MS can be adapted to epilepsy, to meet the needs of this patient population.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to systems, methods and apparatuses/device for tracking a body or portions thereof. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

1. A system for determining a prognosis of a neurological disease in a subject, the system comprising a plurality of sensors for obtaining measurements of the subject, a computational device comprising a processor and memory, wherein instructions are stored on said memory for execution by said processor, wherein said computational device receives sensor data from said sensors and analyzes said sensor data to determine one or more states of the subject, wherein said sensor data is analyzed by a machine learning or deep learning engine, wherein said engine comprises one or more machine learning or deep learning algorithms trained to analyze said sensor data and to determine a prognosis of the neurological disease of the subject from said sensor data, wherein said algorithms are executed by said processor according to instructions stored on said memory.

2. The system of claim 1, wherein said processor is configured to perform a defined set of basic operations in response to receiving a corresponding basic instruction selected from a defined native instruction set of codes; said computational device comprising a first set of machine codes stored in the memory and selected from the native instruction set for receiving said sensor data, a second set of machine codes stored in the memory and selected from the native instruction set for processing said sensor data to form processed sensor data; and a third set of machine codes stored in the memory and selected from the native instruction set for executing said one or more machine learning or deep learning algorithms to analyze said processed sensor data.

3. The system of claim 1, wherein said memory stores instructions for enabling said processor to process said sensor data to form processed sensor data, wherein said instructions include transforming said sensor data according to one or more of FFT (Fast Fourier Transform), short FFT, DCT (discrete cosine transform), fast discrete cosine transform, DFT (discrete Fourier Transform), DWT (discrete wavelet transform) or Hilbert space transforms.

4. The system of claim 3, wherein said instructions further comprise instructions for transforming a plurality of different types of sensor data to a consistent Hilbert space to form common Hilbert space data.

5. The system of claim 4, wherein said instructions for executing said one or more machine learning or deep learning algorithms to analyze said processed sensor data further comprise instructions for receiving a plurality of different types of sensor data as said common Hilbert space data and analyzing said common Hilbert space data according to said one or more machine learning or deep learning algorithms.

6. The system of claim 5, wherein said machine learning algorithms comprise one or more of Naive Bayesian algorithm, Bagging classifier, SVM (support vector machine) classifier, NC (node classifier), NCS (neural classifier system), SCRLDA (Shrunken Centroid Regularized Linear Discriminate and Analysis), Random Forest.

7. The system of claim 6, wherein said deep learning algorithms comprise one or more of a CNN (convolutional neural network), RNN (recurrent neural network), DBN (deep belief network), and GAN (generalized adversarial network).

8. The system of claim 7, wherein said instructions further comprise instructions for denoising said sensor data before analyzing said sensor data.

9. The system of claim 7, wherein said instructions further comprise instructions for receiving medical record data and transforming said medical record data to structured data.

10. The system of claim 9, wherein said instructions further comprise instructions for analyzing said structured data by said one or more machine learning or deep learning algorithms.

11. The system of claim 7, wherein said instructions further comprise instructions for determining a diagnosis for the subject by determining a location of the subject on a diagnostic tree according to said analyzed data.

12. The system of claim 11, wherein said instructions further comprise instructions for converting said data to a unified data structure, mapping said unified data structure information to data characteristics and applying a machine learning model, to determine said location on said diagnostic tree.

13. The system of claim 12, wherein said instructions further comprise instructions for comparing said location on said diagnostic tree for said subject to locations on said diagnostic tree for additional subjects, wherein said prognosis for said additional subjects is known, to determine said prognosis for the subject.

14. The system of claim 7, wherein said instructions further comprise instructions for determining a treatment path for the subject according to a diagnosis and said prognosis.

15. The system of claim 7, wherein the neurological disease is selected from the group consisting of stroke, dementia, epilepsy, multiple sclerosis, neuroinfection, ALS, Parkinson's disease and traumatic brain injuries.

16. The system of claim 7, further comprising adjusting said treatment of the subject according to a plurality of additional sensor measurements.

17. The system of claim 7, wherein said prognosis of the subject is determined according to one or more of facial expression or micro-expression; EEG (electroencephalography) activity; voice related features; determining the position and/or movement of the subject.

18. The system of claim 7, wherein said sensor data relates to one or more of video data, audio data, EMG (electromyography) data, EEG data, ECG, TOF (time of flight) data, other optical data, depth data, and imaging modality data.

19. The system of claim 18, wherein said imaging modality data comprises one or more of PET, MRI, fMRI, SPECT and CAT data.

20. The system of claim 7, wherein said computational device further comprises a data translation layer for translating said data for ingestion by said engine.

21. The system of claim 20, wherein said data translation layer combines a plurality of different data types from different sensors into a consistent multi-dimensional vector space.

22. The system of claim 21, wherein said data translation layer denoises said sensor data before combining said plurality of different data sensor types.

23. The system of claim 22, wherein said data in said consistent multi-dimensional vector space is fed into a single machine learning or deep learning algorithm.

24. The system of claim 23, wherein said data in said consistent multi-dimensional vector space is fed into a plurality of machine learning or deep learning algorithms.

25. The system of claim 24, wherein results from said plurality of machine learning or deep learning algorithms are combined through a single machine learning or deep learning algorithm to determine said prognosis.

26. The system of claim 7, wherein said machine learning or deep learning algorithm comprises a CNN (convolutional neural network) receiving a plurality of data types in a plurality of convolutional and pooling layers, featuring a single connected layer for determining said prognosis of the subject.
